SEQUENCE LISTING (38 sequences)

SEQ ID NO: 1 - DNA, 20 bp, Artificial Sequence
  synthetic primer
aaagtcgcgcacggccttca

SEQ ID NO: 2 - DNA, 20 bp, Artificial Sequence
  synthetic primer
gccagcgccacgatgtccat

SEQ ID NO: 3 - DNA, 19 bp, Artificial Sequence
  synthetic primer
acccctgcctaaccacatc

SEQ ID NO: 4 - DNA, 21 bp, Artificial Sequence
  synthetic primer
gcggcaaagaatcttggagac

SEQ ID NO: 5 - DNA, 20 bp, Artificial Sequence
  synthetic primer
tacaggtctggtttagcaac

SEQ ID NO: 6 - DNA, 20 bp, Artificial Sequence
  synthetic primer
gatctgacacagcttgtgga

SEQ ID NO: 7 - DNA, 20 bp, Artificial Sequence
  synthetic primer
ttctccccatccctctcgct

SEQ ID NO: 8 - DNA, 21 bp, Artificial Sequence
  synthetic primer
agcctggttgaccttcttcag

SEQ ID NO: 9 - DNA, 20 bp, Artificial Sequence
  synthetic primer
ccgcccttgacaaaaagcaa

SEQ ID NO: 10 - DNA, 20 bp, Artificial Sequence
  synthetic primer
gcgcaggatctttccctctt

SEQ ID NO: 11 - DNA, 21 bp, Artificial Sequence
  synthetic primer
gctaaccctcaccaacctcat

SEQ ID NO: 12 - DNA, 19 bp, Artificial Sequence
  synthetic primer
ggttgctgcactttggtcc

SEQ ID NO: 13 - DNA, 20 bp, Artificial Sequence
  synthetic primer
gcgcgctcctgaaacccgaa

SEQ ID NO: 14 - DNA, 20 bp, Artificial Sequence
  synthetic primer
tcggcgttggtggtcttgcg

SEQ ID NO: 15 - DNA, 20 bp, Artificial Sequence
  synthetic primer
ttcccggctctcacctccgt

SEQ ID NO: 16 - DNA, 20 bp, Artificial Sequence
  synthetic primer
tggcctgcaccagtgtgtgt

SEQ ID NO: 17 - DNA, 21 bp, Artificial Sequence
  synthetic primer
cacacctcgggctcggcataa

SEQ ID NO: 18 - DNA, 21 bp, Artificial Sequence
  synthetic primer
caggccgcagagagcatccag

SEQ ID NO: 19 - DNA, 23 bp, Artificial Sequence
  synthetic primer
ccccttcattgacctcaactaca

SEQ ID NO: 20 - DNA, 22 bp, Artificial Sequence
  synthetic primer
ttgctgatgatcttgaggctgt

SEQ ID NO: 21 - DNA, 21 bp, Artificial Sequence
  synthetic primer
ccatgctgctggctgtgggat

SEQ ID NO: 22 - DNA, 25 bp, Artificial Sequence
  synthetic primer
agcagtacaacacaggtgctcttgc

SEQ ID NO: 23 - DNA, 20 bp, Artificial Sequence
  synthetic primer
agggccacggagatgagcct

SEQ ID NO: 24 - DNA, 20 bp, Artificial Sequence
  synthetic primer
ggtcccccggtccctacaca

SEQ ID NO: 25 - DNA, 21 bp, Artificial Sequence
  synthetic primer
gtccagaacctcccctactcc

SEQ ID NO: 26 - DNA, 21 bp, Artificial Sequence
  synthetic primer
cgaaaaccggagtcggaactt

SEQ ID NO: 27 - DNA, 19 bp, Artificial Sequence
  synthetic primer
cccacactacaccgaccag

SEQ ID NO: 28 - DNA, 21 bp, Artificial Sequence
  synthetic primer
ggccataatagggtcctgagg

SEQ ID NO: 29 - DNA, 21 bp, Artificial Sequence
  synthetic primer
ggaggcggaaagccagcgaga

SEQ ID NO: 30 - DNA, 21 bp, Artificial Sequence
  synthetic primer
ctgggctctctgccgcggtta

SEQ ID NO: 31 - DNA, 19 bp, Artificial Sequence
  synthetic primer
cgatcctgggtgtgcgtaa

SEQ ID NO: 32 - DNA, 19 bp, Artificial Sequence
  synthetic primer
gaccaagactgtccccgct

SEQ ID NO: 33 - DNA, 21 bp, Artificial Sequence
  synthetic primer
gagctgcaacttggccacgac

SEQ ID NO: 34 - DNA, 21 bp, Artificial Sequence
  synthetic primer
gagacggagaggaattcagac

SEQ ID NO: 35 - DNA, 23 bp, Artificial Sequence
  synthetic primer
aggcagagccaggagtctgggct

SEQ ID NO: 36 - DNA, 23 bp, Artificial Sequence
  synthetic primer
cggagcactccgcccagcagcga

SEQ ID NO: 37 - DNA, 22 bp, Artificial Sequence
  synthetic primer
tcctagccttggagaagcaact

SEQ ID NO: 38 - DNA, 21 bp, Artificial Sequence
  synthetic primer
cagtgactggagcagagagaa

(19) United States
(12) Patent Application Publication
(10) Pub. No.: US 2015/0299659 A1
(43) Pub. Date: Oct. 22, 2015

(54) MUSCLE CELL PRODUCTION

(71) Applicant: Monash University, Clayton (AU)

(72) Inventors: Tiziano Barberi, San Antonio, TX (US); Bianca Borchin, San Antonio, TX (US)

(21) Appl. No.: 14/601,142

(22) Filed: Jan. 20, 2015

(30) Foreign Application Priority Data
Feb. 11, 2014 (AU) 2014900406

(51) Int. Cl. C12N 5/077; A61K 35/34

(52) U.S. Cl. CPC C12N 5/0658; A61K 35/34; C12N 2501/415; C12N 2501/115; C12N 2501/727; C12N 2506/03

(57) ABSTRACT

The present disclosure relates to production of muscle cell precursors. In particular, the present disclosure relates to an in vitro method of producing skeletal muscle precursor cells from pluripotent stem cells. The present disclosure also further relates to the production of skeletal muscle cells (myoblasts and myocytes) from differentiated stem cells. The present disclosure also relates to the use of these cells in various research applications as well as their use in the treatment of skeletal muscle diseases or disorders.

INCORPORATION BY REFERENCE

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

The sequence listing forms part of this disclosure, the entire contents of which are incorporated by reference herein.

### CROSS REFERENCE TO RELATED APPLICATION

This application claims priority from Australian provisional application 2014900406 filed 11 Feb. 2014, the entire contents of which are incorporated by reference herein including all tables, figures and claims.

### FIELD OF THE INVENTION

The present disclosure relates to production of muscle cell precursors. In particular, the present disclosure relates to an in vitro method of producing skeletal muscle precursor cells from pluripotent stem cells. The present disclosure also further relates to the production of skeletal muscle cells (myoblasts and myocytes) from differentiated stem cells. The present disclosure also relates to the use of these cells in various research applications as well as their use in the treatment of skeletal muscle diseases or disorders.

### BACKGROUND OF THE INVENTION

Pluripotent stem cells (PSCs) such as embryonic stem cells (ESCs) and induced PSCs (iPSCs) provide an extraordinary research tool. In vitro, these cells display extensive proliferation and the ability to differentiate into derivatives of all three germ layers. Such characteristics give these cells a remarkable potential for use in cell-based therapies as well as an in vitro model for early human development. PSC differentiation protocols are currently available for a vast number of cell types (Trounson A (2006) Endocr Rev 27:208-219); however, little progress has been made regarding differentiation of PSCs into derivatives of paraxial mesoderm, such as skeletal muscle. The lack of progress probably stems from the limited availability of data about specific inductive signals and their timing of expression required for myogenic induction of paraxial mesoderm.

The appropriate combination of markers for efficient isolation of skeletal muscle precursors also remains to be determined. As such, only a few studies have reported the derivation of skeletal muscle cells from human PSCs (hPSCs), and they mostly utilized an approach that relies on forced transgene expression to induce myogenesis (Darabi et al., (2012) Cell Stem cell 10:610-619, Goudenege et al., (2012) Mol Ther 20:2153-2167, Ryan et al., (2012) Stem Cell Rev 8:482-493). Although a derivation protocol based on the use of genetically modified PSCs can be successful, it does not reflect normal development, does not provide clear information about the identity of the cells generated, and, most importantly, is not suitable for therapeutic purposes or in vitro disease modeling.

Transplantation of highly purified skeletal muscle precursors has been considered a possible option for the treatment of degenerative muscle disorders, such as muscular dystrophy.

A need therefore exists for an efficient method of producing purified skeletal muscle precursor cells and skeletal muscle cells from pluripotent stem cells.

Citation or identification of any document in this application is not an admission that such document is available as prior art to the present disclosure.

### SUMMARY OF THE INVENTION

The present inventors sought to develop a tightly controlled method to direct human pluripotent stem cells (hPSC) through defined developmental events leading to the derivation of committed skeletal muscle precursor cells. A simple two-step in vitro differentiation protocol was used in which paraxial mesoderm was induced by treating hPSCs with a WNT agonist, a small-molecule glycogen synthase kinase-3 inhibitor. In addition to paraxial mesoderm induction, canonical WNT activation acted as a dorsalizing agent, promoting the generation of dorsal neuroepithelial and neural crest cells. Expansion of the myogenic compartment was achieved by addition of a fibroblast growth factor. Flow cytometry analysis was then utilised to isolate substantially pure skeletal muscle precursors.

The present disclosure is thus directed to methods involving the induction and purification of skeletal muscle precursor cells. Methods for the induction and purification of skeletal myocytes and myoblasts are also described. The cells have utility in a number of therapeutic and non-therapeutic applications.

The present disclosure provides an in vitro method for producing skeletal muscle precursor cells from pluripotent stem cells (PSCs), comprising contacting the stem cells with a canonical WNT pathway agonist, more specifically a glycogen synthase kinase 3 beta (GSK3β) inhibitor, for a time and under conditions sufficient to induce differentiation of the stem cells into skeletal muscle precursor cells. In a particular example, the stem cells are induced to paraxial mesoderm. In another example, the pluripotent stem cells are isolated human pluripotent stem cells, inducible pluripotent stem cells or a pluripotent stem cell line. In another example, the pluripotent stem cells are not embryoid bodies.

In one example, the cells are contacted with the WNT agonist or GSK3β inhibitor in the absence of any additional factors. In another example, the cells may be contacted with the WNT agonist or GSK3β inhibitor for at least 4 days. In a further example, the cells may be contacted with the WNT agonist or GSK3β inhibitor for up to 4 days. In yet a further example, the cells are contacted with the WNT agonist or GSK3β inhibitor for about 4 days. In another example, the concentration of WNT agonist or GSK3β inhibitor is about 3 μM. In another example, the concentration of WNT agonist or GSK3β inhibitor is up to 3 μM. In yet a further example, the concentration of WNT agonist or GSK3β inhibitor is greater than 1 μM.

In another example, the WNT agonist or GSK3β inhibitor is replaced with a fibroblast growth factor (FGF). In a further example the WNT agonist or GSK3β inhibitor is replaced with FGF after about 4 days. In a still further example, the FGF is removed after about 14 days. In yet another example, the FGF is FGF2.

In a particular example, the method according to the present disclosure comprises the steps of:
i) contacting the stem cells with the WNT agonist or GSK3β inhibitor as described herein;
ii) contacting the stem cells with a fibroblast growth factor (FGF);
iii) allowing the cells to differentiate into stem cell progeny.

wherein step (i) precedes step (ii) by up to 4 days.

The cells may be contacted with the WNT agonist or GSK3β inhibitor according to step (i) for time and under conditions sufficient to induce paraxial mesoderm specification of the PSCs.

In a particular example, the WNT agonist or GSK3β inhibitor is removed prior to the addition of FGF. In another example, the FGF is FGF2. In a still further example, the concentration of FGF2 is about 20 ng/ml. In another example, the cells are contacted with FGF or FGF2 for up to14 days after removal of the WNT agonist or GSK3β inhibitor. The cells may be contacted with FGF or FGF2 for about 14 days following removal of the WNT agonist or GSK3β inhibitor. In another example, the FGF or FGF2 is withdrawn/removed after about 14 days of contact with the stem cells.

The method according to the present disclosure may further comprise iv) culturing the stem cells in the absence of any factor (e.g. differentiation factor) for a time and under conditions to drive their differentiation towards the muscle cell lineage. Methods to determine cell differentiation will be familiar to persons skilled in the art. In one example, the cells are differentiated for a time to permit the expression of one or more muscle specific markers individually or collectively in the differentiated stem cell progeny including SIX1, SIX4, PAX3, PAX7, LBX1, MYF5 and MYOG. The cells may also express one or more other muscle specific markers known in the art, for example sarcomeric myosin (MF20) or MYOD. In a particular example, the differentiated stem cell progeny are PAX-3⁺/PAX-7⁺ skeletal muscle precursor cells.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers, and that, notwithstanding that such numbers or combinations of markers or groups of markers may not be specifically listed herein, the accompanying claims may define such combinations or sub-combinations separately and divisibly from any other combination of markers or groups of markers. In one example, expression of the markers is determined by quantitative PCR (qPCR). In another example, expression is determined by immunohistochemistry. In yet another example, expression is determined by flow cytometry.

Preferably, the differentiated stem cell progeny express one or more muscle specification genes selected from the group consisting of SIX1, SIX4, PAX3, PAX7, LBX1, MYF5 and MYOD. In another example, the differentiated stem cell progeny express PAX3 and/or PAX7 In yet another example, at least 18% of the differentiated stem cell progeny express PAX3 and/or PAX7. In another example, at least 20%, 25%, 30%, 35%, 40%, 45%, 50%, 55%, 60%, 65%, 70%, 75%, 80%, 85%, or 90% of the differentiated stem cell progeny express PAX3 and/or PAX7.

In another example, the stem cells are cultured for at least 21 days, at least 25 days, at least 30 days or at least 35 days, wherein day 0 represents the addition of WNT agonist or GSK3β inhibitor according to the method. In a particular example, the stem cells are cultured for about 35 days, wherein day 0 represents the addition of WNT agonist or GSK3β inhibitor according to the method.

In one example, the GSK3β inhibitor according to the present disclosure is CHIR 99021 (Cohen P et al. (2004) Nat Rev Drug Disc 3:479-487).

In a particular example, the stem cells are cultured in serum-free medium. In another example, the cells are cultured in the absence of a feeder layer. In another example, the stem cells are cultured on a matrix (e.g. Matrigel).

The pluripotent stem cells (PSC) according to the present disclosure may be inducible pluripotent stem (iPS) cells, embryonic stem (ES) cells, primate pluripotent stem (pPS) cells, stimulus-triggered acquisition of pluripotency, or “STAP” cells (Obokata H et al (2014) Nature vol 505:641), an embryonic stem cell line or combinations of any one of these. In one example, the pluripotent stem cells are human. In one example, the PSC is WA-09 [H9], Melt or HES3. In one example, the ipS cells are PDL-iPS cells.

In one example, the stem cells are isolated.

The present disclosure also comprises the further step of iv) purifying skeletal muscle cell populations from the differentiated stem cell progeny. Preferably the method comprises purifying myoblasts and mature myocytes from the differentiated stem cell progeny, comprising
(i) contacting the differentiated stem cell progeny with a ligand that binds to HNK and a ligand that binds to muscle specific nicotinic acetyl choline receptor (ACHR); and
(ii) separating HNK⁻/ACHR⁺ cells from the remaining cells.

This method provides a single-antigen strategy for the direct isolation and purification of mature skeletal myocytes.

In one example, at least 20% of the purified cells comprise a mixture of myoblasts and mature myocytes. In another example, at least 30%, 40%, 50%, 60%, 70%, 80%, 90%, 95%, 100% of the purified cells comprise a mixture of myoblasts and mature myocytes. In a further example, the step of contacting the cells with HNK may be carried out separately from the step of contacting the cells with ACHR, whereby the cells are first sorted on the basis of HNK/CD57 expression (lack of HNK expression) prior to sorting on the basis of ACHR expression.

In one example, the ligands are labelled antibodies.

In one example, the ligands specifically bind.

In one example, separating the cells comprises flow cytometry.

The ACHR⁺/HNK⁻ cells may be cultured for a further period of time sufficient to allow fusion of myocytes into multinucleated myotubes. In one example, the cells are cultured for at least 20 days. In another example, the ACHR⁺/HNK⁻ cells are cultured on fibronectin/laminin-coated plates. In another example, the ACHR⁺/HNK⁻ cells are myosin heavy chain 2a positive (MYCH2+). In another example the cultured cells express myogenin and MF20. In another example, the ACHR⁺/HNK⁻ cells are PAX3⁺ and/or PAX7⁺.

The present disclosure alternatively comprises the further step (iv) of purifying skeletal muscle precursor cells from the differentiated stem cell progeny. Preferably the method comprises purifying skeletal muscle precursor cells, the method comprising
(i) contacting the differentiated stem cell progeny with a ligand that binds to HNK and a ligand that binds to muscle specific nicotinic acetyl choline receptor (ACHR);
(ii) contacting the HNK⁻/ACHR⁻ cells with a ligand that binds to C-MET and a ligand that binds to CXCR4; and
(iii) separating C-MET⁺/CXCR4⁻, C-MET⁺/CXCR4⁺ and C-MET⁻/CXCR4⁺ cells from the remaining cells.

In one example, the step of contacting the cells with ligands that binds to C-MET and CXCR4 is carried out after the differentiated stem cell progeny have been separated on the basis of lack of expression of HNK and ACHR. In a further example, the NHK−/ACHR− cells are selected on the basis of CXCR4 expression prior to selection based on expression of C-MET.

Preferably, the method comprises separating C-MET⁺/CXCR4⁻ and C-MET⁺/CXCR4⁺ cells.

In one example, the ligand specifically binds to a cell surface marker.

In one example, the purification method comprises flow cytometry.

In another example, the purification method is carried out after the stem cells have been cultured for about 35 days, wherein day 0 represents the addition of a WNT agonist or GSK3β inhibitor. In another example, the purification is carried out after the cells have been cultured for at least 25, or at least 30 days.

In one example, at least 85% of C-MET⁺/CXCR4⁻ cells express PAX-7. In another example, at least 87%, 88%, 89%, 90%, 91%, 92%, 93%, 94%, 95%, 96%, 97%, 98% or 99% of the C-MET⁺/CXCR4⁻ cells express PAX7. In another example, at least 96% of C-MET⁺/CXCR4⁻ cells express PAX3. In another example, at least 97%, 98% or 99% of the C-MET⁺/CXCR4⁻ cells express PAX3. In another example, at least 95% of C-MET⁺/CXCR4⁺ cells express PAX7. In another example, at least 96%, 97%, 98% or 99% of C-MET⁺/CXCR4⁺ cells express PAX7. In another example, at least 97% of C-MET⁺/CXCR4⁺ cells express PAX3. In another example, at least 98% or 99% of the C-MET⁺/CXCR4⁺ cells express PAX3. In yet another example, at least at least 98% or at least 99% of the cells expressing PAX7 also express PAX3.

In one example, the skeletal muscle precursor cells are PAX3⁺/PAX7⁺. In a further example, the skeletal muscle precursor cells do not express neural cell markers. In another example the skeletal muscle precursor cells do not express SOX1. In another example, the cell populations (C-MET⁺/CXCR4⁻ and C-MET⁺/CXCR4⁺) comprise at least 98% skeletal muscle precursor cells.

The C-MET+/CXCR4− and C-MET+/CXCR4+ cells may be further differentiated to mature skeletal muscle cells. In one example, the differentiated cells express MYF5. In another example, the differentiated cells express MYF5, MYOG and MF20.

In one example, the step of generating differentiated stem cell progeny according to any method as described herein comprises
(a) contacting pluripotent stem cells with a WNT agonist or GSK3β inhibitor for about 4 days;
(b) contacting the stem cells with a fibroblast growth factor (FGF) for about 14 days;
(c) culturing the cells for a further period of at least about 10 days in the absence of any factor;

wherein step (a) precedes step (b).

In one example, according to step c) the cells are cultured for a further period of at least 10 days. In one example, according to step c) the cells are cultured for a further period of between 10 and 17 days. In another example, according to step c), the cells are cultured for a period of 17 days.

In a particular example, the WNT agonist or GSK3β inhibitor is removed prior to the addition of FGF. In a further example the FGF is FGF2.

In yet another example, the cultured cells are further purified according to the methods described herein. In a further example, the purified cells may be further cultured.

The method according to the present disclosure is preferably conducted under cell culture conditions. In one example, the cell culture conditions comprise plating the pluripotent stem cells in cell culture dishes. In a further example, the cell culture dishes comprise a suitable medium for maintaining the PSCs. In yet another example, the cells are plated onto a suitable matrix (e.g Matrigel). The cell culture may be upscaled to a suitable bioreactor or equivalent cell culture apparatus.

The present disclosure also provides purified skeletal muscle precursor cells or myoblasts and mature myocytes produced by a method described herein.

The present disclosure also provides a composition comprising purified skeletal muscle precursor cells as described herein, myoblasts and mature myocytes as described herein, or cultured ACHR+/HNK− cells as described herein, together with a pharmaceutically acceptable carrier or excipient. In a further example, the present disclosure provides a composition comprising purified PAX3⁺/PAX7⁺ skeletal muscle precursor cells. In another example, the present disclosure provides a composition comprising purified HNK⁻/ACHR⁺ myoblasts and mature myocytes.

In one example, the composition is a pharmaceutical composition. Preferably, the cells are provided in a therapeutically effective amount.

The present disclosure also provides use of purified skeletal muscle precursor cells as described herein or myoblasts and mature myocytes as described herein for in vitro screening of agent(s) which are capable of modifying the function of the cells. In a further example, the cells are used to screen for agent(s) which are capable of modifying insulin resistance in the cells.

The present disclosure also provides the use of purified skeletal muscle precursor cells as described herein or myoblasts and mature myocytes as described herein for disease modeling, drug development and/or toxicity studies.

The present disclosure also provides use of purified skeletal muscle precursor cells as described herein or myoblasts and mature myocytes as described herein in regenerative medicine.

The present invention also provides use of purified skeletal muscle precursor cells as described herein or myoblasts and mature myocytes as described herein for treating a skeletal muscle injury, disease or disorder in a subject in need thereof.

The present disclosure also provides purified skeletal muscle precursor cells as described herein, or myoblasts and myocytes as described herein, or a composition as described herein, for use in treating a treating a skeletal muscle injury, disease or disorder in a subject in need thereof.

The present disclosure also provides a method of treating a skeletal muscle disease or disorder in a subject in need thereof, comprising administering to the subject purified skeletal muscle precursor cells as described herein, myoblasts and mature myocytes as described herein, cultured ACHR⁺/HNK⁻ cells as described herein, or a composition comprising such cells.

The present disclosure also provides use of purified skeletal muscle precursor cells as described herein, myoblasts and mature myocytes as described herein, cultured ACHR+/HNK− cells as described herein, or a composition as described herein in the manufacture of a medicament for treating a skeletal muscle disease or disorder. Preferably, the cells are provided in a therapeutically effective amount.

In one example according to any use or method of treatment described herein, the stem cells are autologous or allogeneic. If allogenic cells are used, it is preferable that the pluripotent stem cells (which would preferably be iPS cells) have the same or substantially the same type of HLA. The stem cells are preferably derived from a mammal. In a further example, the stem cell are human or human-derived.

The disease or disclosure according to the present disclosure may be selected from the group consisting of muscular dystrophy (e.g. Duchenne's muscular dystrophy (DMD), Becker type muscular dystrophy, congenital muscular dystrophy, limb-girdle muscular dystrophy, myotonic muscular dystrophy and the like), hereditary myopathies such as congenital myopathy, distal myopathy and mitochondrial diseases, non-hereditary myopathies such as multiple myositis, dermatomyositis and myasthenia gravis, neurogenic muscular diseases, spinal amyotrophy, bulbar amyotrophy and amyotrophic lateral sclerosis.

In one example, the subject according to the present disclosure is human.

### BRIEF DESCRIPTION OF THE ACCOMPANYING DRAWINGS

FIG. 1 CHIR treatment promotes induction of dorsal tissue in differentiating hPSC.

Immunocytochemical analysis of hESC (H9) at day 12 of differentiation under treatment conditions, showing (A) Neural crest marker SOX10 (green), dorsal neural tube/roof plate marker LMX1a (red) and (B) SOX10 (green) and non-neural ectoderm marker AP2α (red) Scale bars: 50 μm. (C) RT-PCR analysis for LMX1a, SOX10 and AP2α expression at day 10 of hESC (HES3) differentiation. SOX10 and LMX1a are expressed only in CHIR (GSK3β inhibitor) treated cells while AP2α expression is detected also in untreated cells.

FIG. 2 Derivation of skeletal muscle from hPSCs.

(A) Schematic diagram summarizing the treatment protocol for the induction of myogenic differentiation from hPSCs. Immunocytochemical detection of (B) Representative fields of PAX3+ and PAX7+ skeletal muscle precursors and (C) MF20+/Myogenin+ mature skeletal myocytes in unsorted cultures at day 35 of hESC (H9) differentiation, under treatment conditions. Scale bar=50 μm. (D) Quantitative analysis of PAX3+/7+ nuclei and MF20+ cells at day 35 of hPSC differentiation (H9, HES3, MEL1, DPL-iPS, n=4) in unsorted cultures.

FIG. 3 Detection of gene transcripts relevant to the acquisition of a myogenic cell fate.

Quantitative polymerase chain reaction (qPCR) analysis showing transcript levels of key muscle development genes from hPSCs (DPL-iPS, H9, MEL1, HES3 n=4) differentiating under treatment conditions vs medium alone. Cells were collected and analysed at three day intervals between days 0 and 30 of hPSC differentiation. The relative expression level of each gene is calibrated to its expression at day 0 (represented on the Y axis). Ct values for each gene are normalized to Ct values of the reference gene, GAPDH. Values represent mean±SEM of four independent experiments. Red dots mark early peaks of PAX3 and PAX7 expression corresponding to the timing of development of early dorsal neural tissues (roof plate/neural crest).

FIG. 4 FACS strategy for the isolation of myogenic cell populations.

Shown is a representative experiment in which hESCs (MEL1) differentiating for 35 days under treatment conditions were sorted based on their HNK, ACHR, CXCR4, and C-MET surface marker expression. The gates in each dot plot designate the cell fraction analyzed for the prospective steps. +/− is indicative of either positive or negative expression of each surface antigen. The myogenic cell populations collected from sorting were as follows: (HNK−/ACHR+), (HNK−/ACHR−/CXCR4−/C-MET+), (HNK−/ACHR−/CXCR4+/C-MET+), (HNK−/ACHR−/CXCR4+/C-MET−). Gate I: HNK− cells were selected to exclude HNK+ neural/neural crest component. Gate II: selection of HNK−/ACHR− cells for myogenic precursor isolation at subsequent steps or direct isolation of HNK−/ACHR+ mature myocytes. Gate III: selection of CXCR4+/− cells. Gate IV: Isolation of myogenic precursor cell populations: HNK−/ACHR−/CXCR4−/C-MET+ from gated CXCR4− cells and HNK−/ACHR−/CXCR4+/C-MET+, HNK−/ACHR−/CXCR4+/C-MET− from gated CXCR4+ cells.

FIG. 5 Characterization of CXCR4−/C-MET+ and CXCR4+/C-MET+ sorted populations.

(A) Cytospin preparations of muscle precursor cell populations CXCR4−/C-MET+ (top) and CXCR4+/C-MET+ (bottom) sorted at day 35 of hESC (HES3) differentiation. Cells were cytospun on glass slides and analyzed by immunocytochemistry for myogenic stem cell markers PAX3 (green) and PAX7 (red) immediately following sorting. Each dot represents one nucleus as confirmed by DAPI counterstaining. (B) Immunostaining of replated muscle precursors CXCR4−/C-MET+ (left) and CXCR4+/C-MET+ (right) (from hESCs-MEL1) at days 3, 6 and 9 of post-sorting cultures showing progression towards a muscle terminal differentiation phenotype. (C) RT-PCR analysis of skeletal muscle precursor genes (PAX3, PAX7, LBX1) and neural gene (SOX1) in all sorted populations (from DPL-iPS) derived under treatment conditions. Abbreviations: myogenin (MYOG); sarcomeric myosin (MF20). Scale bars=50 μm.

FIG. 6 Developmental progression of hPSC-derived myogenic cell populations.

Representative immunocytochemical analysis on cytospin preparations of precursor population's CXCR4−/C-MET+ and CXCR4+/C-MET+ at days 23, 25 and 30 of hESC (MEL1) differentiation. Expression of early myogenic regulatory genes SIX4 and PAX3 is detectable as early as day 23, prior to PAX7 expression as expected during myogenic lineage progression. As development proceeds (days 25-30), an increase in PAX7 expression is observed. Scale bars=50 μm.

FIG. 7 Isolation of ACHR+ skeletal myocytes.

(A) Phase contrast image (left) and immunocytochemical analysis for ACHR expression (right) on hESC-derived (MEL1) skeletal myocytes prior to FACS isolation. (B) FACS profile of ACHR+ cell population (from hESC-H9). (C) RT-PCR analysis of mature skeletal muscle marker MYH2 in ACHR negative cells (Neg) and positive cells (ACHR+) (from HES3). (D) Immunocytochemical analysis of hESC-derived ACHR+ myocytes (H9) 24 hours post-sort, expressing mature skeletal muscle proteins, MF20 and MYOG. (E) Phase contrast image showing morphology of ACHR+ myocyte-derived myotubes (from H9) after prolonged cell culture (>20 days). Scale bars=50 μm. Abbreviation: myosin heavy chain 2 (MYH2).

FIG. 8 Quantification of ACHR+ CXCR4−/C-MET+ and CXCR4+/C-MET+ cell populations derived across independent cell lines.

Percentage of myogenic cell populations isolated at day 35 from four hPSC lines differentiated under treatment conditions (CHIR and FGF2). Efficiency of myogenic differentiation was similar across all cell lines. Results shown for each cell population represents n=3 experiments averaged for each of the 4 hPSC lines (DPL iPS, Mel1, H9 and HES3).

FIG. 9 Quantification of ACHR+, CXCR4+/C-MET+, CXCR4−/C-MET+, and CXCR4+/C-MET− cell populations derived from hPSCs differentiated under 4 treatment conditions.

CHIR+FGF2; CHIR only; FGF2 only; Untreated. (A) Percentage of ACHR+ myocytes (I), CXCR4+/C-MET+ (II), CXCR4−/C-MET+ (III) precursors and CXCR4+/C-MET− (IV) mixed population from multiple FACS purification experiments at three different time points. Results shown for each treatment condition represent (n=3) experiments averaged from each of the 4 hPSC lines. (I, II) Fold change difference is observed between CHIR+FGF2 treatment and all other conditions at each time-point. CHIR+FGF2 treatment significantly (p<0.001) improves induction of both cell populations at day 35 of differentiation compared to FGF2 only or untreated cultures. (III, IV) Percentage of cells is independent of treatment, however, cell composition is altered (refer to FIG. 10). (B) Shown is a representative FACS profile of hPSCs (H9) at day 35 of differentiation. The sorted populations are represented as percentage fractions of respective parent population. ACHR+ (top) and CXCR4+/C-MET+ (middle) cell populations are only present in CHIR treated hPSCs while CXCR4+/C-MET− (middle) and CXCR4−/C-MET+ (bottom) cell populations are present under all conditions. ̂ % of ACHR+ gated populations are based on HNK− gated fractions. * % of CXCR4+/C-MET+ and CXCR4+/C-MET− gated populations are based on HNK−/ACHR−/CXCR4+ gated fractions. ^{Λ} % of CXCR4−/C-MET+ gated populations are based on HNK−/ACHR−/CXCR4− gated fractions. Error bars represent the SEM of three or more individual experiments.

FIG. 10 Lack of CHIR treatment during hPSC differentiation results in the absence of a muscle phenotype.

Immunocytochemical analysis from cytospin preparations of CXCR4+/C-MET− (A, B) and CXCR4−/C-MET+ (C, D) sorted cells. (A) Under CHIR+FGF2 treatment, majority of CXCR4+/C-MET− cells were PAX7+ indicating a predominant muscle phenotype. (B) A complete switch towards SOX1 expression is observed in FGF2 only conditions. (C) CXCR4−/C-MET+ cell population derived from CHIR+FGF2 treated hPSCs is composed of highly enriched PAX3+/PAX7+ muscle precursors, (D) PAX3+ and PAX7+ cells are not present under FGF2 alone conditions, with a large number of cells instead expressing the non-neural ectoderm marker AP2α. Scale bars=50 μm. All images from hESC HES3.

### DETAILED DESCRIPTION OF THE INVENTION

### General Techniques and Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

Any example disclosed herein shall be taken to apply mutatis mutandis to any other example unless specifically stated otherwise.

Unless specifically defined otherwise, all technical and scientific terms used herein shall be taken to have the same meaning as commonly understood by one of ordinary skill in the art (e.g., in cell culture, molecular genetics, stem cell differentiation, immunology, immunohistochemistry, protein chemistry, and biochemistry).

Unless otherwise indicated, the stem cells, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, of the designated value. With respect to days, the term “about” refers to +/−1 day.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “allogeneic” refers to tissue, cells or stem cells being genetically different, but deriving from the same species.

As used herein, the term “autologous” refers to tissue, cells or stem cells that are derived from the same subject's body.

The term “muscle specific marker” is understood to mean a protein, glycoprotein or other molecule which is present or displayed on the surface of a cell which serves to identify the cell. It will be also understood by persons skilled in the art to mean that the protein, glycoprotein or other molecule is expressed on the cell surface. A cell surface marker can generally be detected by conventional methods, for example, fluorescence activated cell sorting (FACS) or enzymatic analysis. Cell surface markers can be utilized for positive or negative selection processes. For example, a positive selection marker such as C-MET or CXCR4 is present on the differentiating cells of interest. Markers for negative selection are absent on the differentiating cells or interest but will typically be present on other cells in the sample e.g. neural cells, neural crest cells etc. Negative selection markers may therefore also be utilized in the methods of the present disclosure. For example, a negative selection marker such as HNK-1 or muscle specific nicotinic acetylcholine receptor (ACHR) is not present on muscle cell precursors but is present on other cells in the sample.

As used herein, the term “skeletal muscle injury, disease or disorder” refers to an injury disease or disorder related to the muscular system. Skeletal muscle injury, disease and/or disorders include, but are not limited to, direct trauma, laceration, abrasion, bruising, crush injury, contusion injury by short or long impact, tear, strain and/or sprain, muscle injury incurred by cutting or dissection of, or surgical incision into, skeletal muscle, such as muscle injuries that occur during operative or surgical intervention, also known as myotomy, sport injuries, such as torn muscles or torn muscle fibres, and/or injuries occurring from stretching or overstretching muscles and/or injuries incurred during surgery or operation around the spinal cord and/or vertebral column, in addition to rotator cuff ruptures, muscular dystrophy, hereditary myopathies such as congenital myopathy, distal myopathy and mitochondrial diseases, non-hereditary myopathies such as multiple myositis, dermatomyositis and myasthenia gravis, neurogenic muscular diseases, spinal amyotrophy, bulbar amyotrophy and amyotrophic lateral sclerosis etc.

As used herein, the term “flow cytometry”, is understood to involve the separation of cells in a liquid sample. Generally the purpose of flow cytometry is to analyse the separated cells for one or more characteristics thereof. A fluid sample is directed through an apparatus such that a liquid stream passes through a sensing region. The cells pass the sensor one at a time and are categorized based on size, refraction, light scattering, opacity, roughness, shape, fluorescence, etc. In the context of the present disclosure, the term “flow cytometry” is also understood to encompass cell sorting (fluorescence activated cell sorting).

As used herein, the term “isolated” or “purified” is intended to refer to a cell, isolatable or purified from other components. An isolated cell refers to a cell free from the environment in which it may naturally occur. The isolated cell may be purified to any degree relative to its naturally-obtainable state.

The term “pluripotency” and “pluripotent stem cells” is taken to mean that such cells have the ability to differentiate into all types of cells in an adult organism.

As used herein, the term “inducible pluripotent stem cell (iPS)” or “induced pluripotent stem cell” is understood to mean a pluripotent stem cell artificially derived from a non-pluripotent cell, typically an adult somatic cell, by inducing forced expression of specific genes. The term encompasses pluripotent cells, that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. By having the potential to become iPS cells, it is meant that the differentiated somatic cells can be induced to become, i.e. reprogrammed to become, iPS cells. In other words, the somatic cell can be induced to re-differentiate so as to establish cells having the morphological characteristics, growth ability and pluripotency of pluripotent cells. iPS cells have an hESC-like morphology, growing as flat colonies with large nucleo-cytoplasmic ratios, defined borders and prominent nucleoli. In addition, iPS cells express one or more key pluripotency markers by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, pluripotent cells are capable of forming teratomas.

As used herein, the term “specifically binds” shall be taken to mean that the ligand that binds to a cell surface marker e.g. antibody or protein comprising an antibody variable region reacts or associates more frequently, more rapidly, with greater duration and/or with greater affinity with a particular cell or substance than it does with alternative cells or substances. For example, a ligand that specifically binds to a target protein is a agent that binds that protein or an epitope or immunogenic fragment thereof with greater affinity, avidity, more readily, and/or with greater duration than it binds to unrelated protein and/or epitopes or immunogenic fragments thereof.

As used herein, the term “subject” shall be taken to mean any subject, including a human or non-human subject. The non-human subject may include non-human primates, ungulate (bovines, porcines, ovines, caprines, equines, buffalo and bison), canine, feline, lagomorph (rabbits, hares and pikas), rodent (mouse, rat, guinea pig, hamster and gerbil), avian, and fish. In one example, the subject is a human. Terms such as ‘subject’, ‘patient’ or ‘individual’ are terms that can, in context, be used interchangeably in the present invention. The term “subject” can also refer to any organism which has a skeletal muscle disease or disorder.

As used herein, the term “therapeutically effective amount” shall be taken to mean a sufficient quantity of purified skeletal muscle precursor cells as described herein, myoblasts and mature myocytes as described herein or cultured ACHR+/HNK− cells as described herein, or otherwise CXCR4⁺/C-Met⁺⁻, or CXCR4⁻/C⁻MET⁺ cells as described herein that results in an improvement or remediation of the symptoms of the disease or condition. The skilled artisan will be aware that such an amount will vary depending upon, for example, the particular subject and/or the type or severity or level of disease. The term is not to be construed to limit the present disclosure to a specific quantity, e.g. weight or number of cells, rather the present disclosure encompasses any number of cells sufficient to achieve the stated result in a subject.

As used herein, the terms “treating”, “treat” or “treatment” include administering a therapeutically effective amount of of purified skeletal muscle precursor cells as described herein, myoblasts and mature myocytes as described herein or cultured ACHR+/HNK− cells as described herein, or otherwise CXCR4+/C-MET+−, or CXCR4−/C-MET+ cells as described herein that results, or composition comprising such cells, sufficient to reduce or eliminate at least one symptom of a specified disease or condition.

As used herein the term “positive or (+)” (e.g. CXCR4⁺ or C⁻MET⁺) or “negative or (−)” (e.g. HNK-1⁻) as used herein refers to expression of the cell surface marker compared with a suitable isotype matched control. A cell that is referred to as being “positive” for a given marker may express either a low (lo or dim) or a high (bright, bri) level of that marker depending on the degree to which the marker is present on the cell surface, wherein the terms relate to intensity of fluorescence or other marker used in the sorting process of the cells. The distinction of lo and bri will be understood in the context of the marker used on a particular cell population being sorted. A cell that is referred to as being “negative” for a given marker is not necessarily completely absent from that cell. This term means that the marker is expressed at a relatively very low level by that cell, and that it generated a very low signal when detectably labeled or is undetectable above background levels, e.g., levels detected using an isotype control antibody.

While not wishing to be bound by theory, it is proposed that “bright” cells express more of the target marker protein (for example the antigens recognized by (e.g. CXCR4 and/or C-MET) than other cells in the sample. Typically, positive expression of any given marker will be at least one log magnitude greater, preferably two log magnitude higher expression of the surface marker compared with the isotype matched negative control.

As used herein, a “WNT agonist” is understood as referring to any molecule which activates the WNT signaling pathway. In particular, such activation promotes paraxial mesoderm specification of PSCs. WNT agonists are known in the art and are also commercially available. Examples include, but are not limited to 681665 Wnt agonist (EMD Millipore), Wnt agonist sc-222416 (Santa Cruz Biotechnology), or small molecule agonists as described, for example in Liu Jet al (2005) Angew Chem Int Ed Engl 18; 44(13):1987-90.

### Stem Cells and Pluripotent Cells

A “pluripotent cell” is preferably one which is derived from any kind of embryonic tissue (fetal or pre-fetal tissue) and has the characteristics of being capable under appropriate conditions of producing progeny of different cell types that are derivatives of all the three germinal layers (endoderm, mesoderm and ectoderm), according to standard tests, such as the ability to form a teratoma in 8-12 week old SCID mice, or the ability to form identifiable cells of all three germ layers in tissue culture.

Stem cells of interest according to the present disclosure include embryonic cells of various types, exemplified by human embryonic stem (hES) cells, described by Thomson et al., (Science 282:1145, 1998); embryonic stem cells from other primates, such as Rhesus stem cells (Thomson et al., Proc. Natl. Acad. Sci USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998). These cell types may be provided in the form of an established cell line, or they may be obtained directly from primary embryonic tissue and used immediately for differentiation. Examples of cell lines include those listed in the NIH Human Embryonic Stem Cell Registry, e.g. hESBGN-01, hESBGN-02, hESBGN-03, hESBGN-04 (BresaGen, Inc), HES-1, HES-2 (Reubinoff et al., Nat Biotechnol. 18:399-404, 2000), HES-3 (Mummery C et al., J Anat 200:233-242, 2002; Passier R et al., Stem Cells 23:772-780, 2005), HES-4, HES-5, HES-6 (ES Cell International), Miz-hES1 (MizMedi Hospital-Seoul National University), HSF-1, HSF-6 (University of California at San Francisco); and H1, H7, H9, H13, H14 (Wisconsin Alumni Research Foundation (WiCell Research Institute)).

Also included in the scope of the present disclosure are lineage committed stem cells, such as mesodermal stem cells and other early skeletal muscle cells (Reyes et al. (2001) Blood 98:2615; Eisenberg & Bader (1996) Cic Res. 78(2):205).

Other types of pluripotent cells are also included in the term. Any cells of primate (including human) origin that are capable of producing progeny that are derivatives of all three germinal layers are included, regardless of whether they were derived from embryonic tissue, fetal tissue, or other sources. In example, the stem cells used in the present method are WA-09 [H9], Melt HES3 and/or PDL-iPS (Yoshiaki N et al (2012) Histochemistry & Cell Biology vol 137 (6):719-732)

### Embryonic Stem Cells

ES cells are considered to be undifferentiated when they have not committed to a specific differentiation lineage. Such cells display morphological characteristics that distinguish them from differentiated cells of embryo or adult origin. Undifferentiated ES cells are easily recognised by those skilled in the art, and typically appear under the microscope as colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection.

Embryonic stem cells have been isolated from blastocyts of members of the primate species (Thomson et al., Proc. Natl. Acad. Sci USA 92:7844, 1995). Human embryonic stem (hES) cells can be prepared from human blastocyst cells using the techniques described by Thomson et al., (U.S. Pat. No. 5,843,780; Science 282:1145, 1998) and Reubinoff et al., (Nature Biotech. 18:399, 2000). Equivalent cell types to hES cells include their pluripotent derivatives, such as primitive endoderm-like (EPL) cells, described in WO 01/51610.

Briefly, human blastocytes are obtained from human in vivo preimplantation embryos. Alternatively, in vitro fertilised (IVF) embryos can be used, or one cell human embryos can be expanded to the blastocyst stage (Bongso et al., Human Reprod 4:706, 1989). Embryos are cultured to the blastocyst stage in G1.2 and G2.2 medium. The zona pellucida is removed from the developed blastocyts by brief exposure to pronase. The inner cell masses are isolated (for example by immunosurgery using rabbit anti-human spleen cell antiserum). The intact inner cell mass is plated on mEF feeder cells (U.S. Pat. No. 5,843,780), human feeder cells (US 2002/0072117), or in a suitable feeder-free environment that supports undifferentiated hES cell growth (US 2002/0081724; WO 03/020920). Growing colonies having undifferentiated morphology are dissociated into clumps, and replated.

### Spermatogonial Stem Cells

Spermatogonial stem cells are testis-derived pluripotent stem cells, serving as an origin for spermatogenesis. Spermatogonial stem cells can also be induced to differentiate into cells of various lines in a manner similar to that in ES cells. For example, the cells have properties such that a chimeric mouse can be produced when transplanted into mouse blastocysts (M. Kanatsu-Shinohara et al. (2003) Biol. Reprod., 69: 612-616; K. Shinohara et al. (2004), Cell, 119: 1001-1012). Spermatogonial stem cells are self-replicable in a medium containing a glial cell line-derived neurotrophic factor (GDNF) or spermatogonial stem cells can be obtained by repeated subculture of the cells under culture conditions similar to those for ES cells (Masanori Takebayashi et al., (2008), Experimental Medicine, Vol. 26, No. 5 (Extra Number), pp. 41-46, YODOSHA (Tokyo, Japan)).

### Embryonic Germ Cells

Embryonic germ cells are cells established from primordial germ cells at the prenatal period and have pluripotency similar to that of ES cells. Embryonic germ cells can be established by culturing primordial germ cells in the presence of substances such as LIF, bFGF, and a stem cell factor (Y. Matsui et al. (1992), Cell, 70: 841-847; J. L. Resnick et al. (1992), Nature, 359: 550-551).

### Differentiated Cells

A “differentiated cell” is a cell that has progressed further down the developmental pathway than a cell with which it is being compared. Thus, embryonic stem cells can differentiate to lineage restricted precursor cells (such as a mesodermal stem cell), which in turn can differentiate into other types of precursor cells further down the pathway (such as skeletal muscle precursor cells), and then to an end-stage differentiated cells, which plays a characteristic role in a certain tissue type, and may or may not retain the capability to proliferate further.

The potential of ES cells to give rise to all differentiated cells provides a means of giving rise to any mammalian cells type, and so a range of culture conditions may be used to induce differentiation.

The relative term “differentiating” describes the active process whereby the cell is progressing down the developmental pathways to become more lineage restricted and mature.

Among the differentiated cells of interest are cells not readily grown from somatic stem cells, or cells that may be required in large numbers and hence are not readily produced in useful quantities by somatic stem cells.

### Inducing Differentiation

As will be appreciated by one of skill in the art, stem cell differentiation is not a spontaneous process but rather occurs over time requiring a series of genetic and epigenetic alterations to direct a stem cell towards a particular differentiated state. Accordingly, contacting stem cells with particular factors that promote differentiation may not immediately result in development of a differentiated cell phenotype. For example, stem cells may require a priming or induction step that directs a stem cell towards a particular differentiated state. It is envisaged that such a priming step does not provide a fully differentiated cell but rather directs or commits a stem cell to a particular differentiated state. For example, stimulating the canonical WNT pathway or inhibiting GSK3β is seen as an induction/priming step, where during subsequent culture of the cells, results in the stem cells differentiating into particular cell types. In an example, these cell types include myoblasts, mature myocytes, muscle precursors and/or neural cells.

### Skeletal Muscle Precursor Cells

The term “precursor cell” covers a cell that has a tendency to differentiate into a specific type of cell, but is already more specific than a stem cell and is pushed to differentiate into its “target” cell. For example a muscle precursor cell has the capacity to differentiate into a skeletal muscle cell. In this example, a muscle precursor cell may express particular muscle specific differentiation markers such as SIX1, SIX4, PAX3, PAX7 and/or the migratory skeletal muscle precursor marker LBX1. In an example, a muscle cell precursor may be PAX3⁺ or PAX7⁺. In another example a muscle cell precursor may be PAX3⁺ and PAX7⁺.

The term “muscle” covers a multitude of cell types, all specialized for contraction but in other respects dissimilar. A contractile system involving actin and myosin is a basic feature of animal cells in general, but muscle cells have developed this apparatus to a high degree. Mammals possess four main categories of cells specialized for contraction: skeletal muscle cells, heart (cardiac) muscle cells, smooth muscle cells, and myoepithelial cells. These differ in function, structure, and development. Although all of them generate contractile forces by means of organized filament systems based on actin and myosin, the actin and myosin molecules employed are somewhat different in amino acid sequence, are differently arranged in the cell, and are associated with different sets of proteins to control contraction. The present invention relates to the production of skeletal muscle precursor cells and/or skeletal muscle cells.

Skeletal muscle cells are responsible for practically all movements that are under voluntary control. These cells can be very large (2-3 cm long and 100 μm in diameter in an adult human) and are often referred to as muscle fibers because of their highly elongated shape. Each one is a syncytium, containing many nuclei within a common cytoplasm. The other types of muscle cells are more conventional, generally having only a single nucleus. The term “skeletal muscle cell” is understood to cover all cells committed to the skeletal muscle cell fate, including skeletal muscle precursors, myoblasts, myocytes and myotubes.

The pluripotent/embryonic stem cell derived skeletal muscle cells and their precursors typically have at least one of the following skeletal muscle cell specific markers:

MyoD, is a bHLH (basic helix loop helix) transcription factor belonging to a family of proteins known as myogenic regulatory factors (MRFs). This family includes MyoD, Myf5, myogenin, and MRF4 (Myf6). MyoD is one of the earliest markers of myogenic commitment.

Myf5, is a a bHLH (basic helix loop helix) transcription factor with a key role in regulating muscle differentiation.

Pax3,

Pax7,

Myogenin (MYOG), is a bHLH (basic helix loop helix) transcription factor that is required for the fusion of myogenic precursor cells to either new or previously existing fibres during the process of differentiation in myogenesis.

myosin heavy chain,

NCAM,

Desmin,

SkMAct,

MF20,

M-Cadherin,

Fgfr4,

VCAME1,

C-MET,

CXCR4,

AchR

HNK-1,

Tissue-specific markers can be detected using any suitable immunological technique—such as flow immunocytometry or affinity adsorption for cell-surface markers, immunocytochemistry (for example, of fixed cells or tissue sections) for intracellular or cell-surface markers, Western blot analysis of cellular extracts, and enzyme-linked immunoassay, for cellular extracts or products secreted into the medium. Antibodies that distinguish skeletal muscle cell markers like CPAX3 and CPAX7 from other isoforms are available commercially from suppliers such as those outlined in the Examples.

Expression of tissue-specific gene products can be detected at the mRNA levels by Northern blot analysis, dot-blot hybridization analysis, or by reverse transcriptase initiated polymerase chain reaction (RT-PCR) using sequence-specific primers in standard amplification methods using publicly available sequence data (GenBank). Expression of tissue-specific markers as detected at the protein or mRNA level is considered positive if the level is at least 2-fold, and preferably more than 10- or 50-fold above that of a suitable control.

### Myoblasts and Myocytes

As described herein, the present disclosure described methods for generating myoblasts and mature skeletal muscle myocytes. The term “myoblast” should be given its ordinary meaning, that is, a muscle progenitor cell that is capable of differentiating into mature muscle cells. Typically, myoblasts will be further matured compared to muscle cell “precursors” described above and will express one or more of PAX7, Myf5, MyoD and myogenin (MYOG). Myocytes are formed from myoblasts and are also referred to as muscle cells. The term “cultured ACHR+/HNK− cells” as used herein refers to cells (myoblasts and myocytes) which have been cultured for a period of time to allow for progressive fusion of myocytes into multinucleated myotubes.

Inhibition of Glycogen Synthase Kinase 3 beta (GSK3β)

Glycogen synthase kinase 3 (GSK3) is a serine/threonine kinase for which two isoforms, α and β, have been identified Woodgett, Trends Biochem. Sci., 16:177-81 (1991). Both GSK3 isoforms are constitutively active in resting cells.

The present method relates to the inhibition of the β isoform of GSK3 (GSK3β) in stem cells to promote differentiation of the stem cells into skeletal muscle cells precursors and/or myoblasts and myocytes. In the context of the present invention, the term ‘inhibitor’ is a substance that reduces or suppresses the activity of another substance such as a protein. Accordingly, it is envisaged that any substance that decreases the activity of GSK3β is encompassed by the term, ‘inhibitor of glycogen synthase kinase 3 beta (GSK3 β)’. In an example, the GSK3β inhibitor used in the present method decreases or inhibits the activity of GSK3β sufficiently to activate, up regulate or increase the activity of canonical WNT signaling.

Suitable GSK3β inhibitors useful in the present disclosure are described in patent applications WO 03/004472, WO 03/055492, WO 03/082853, WO 06/001754, WO 07/040436, WO 07/040438, WO 07/040439 and WO 07/040440, WO08/002244 and WO08/002245, which are hereby incorporated by reference.

Preferably, the GSK3β inhibitor is CHIR 99021 (Stemgent). Other suitable GSK3β inhibitors useful in the present disclosure such as AR A014418, SB 216763, SB 415286, bis-7-azaindolylmaleimides, CHIR 98023, CHIR 98014 are described in Cohen P et al. (2004) Nat Rev Drug Disc 3:479-487 and are hereby incorporated by reference.

In the present method, stem cells are contacted with an inhibitor GSK3β. It is considered that terms such as ‘contacting’, ‘exposing’ or ‘applying’ are terms that can, in context, be used interchangeably in the present disclosure. The term contacting, requires that the GSK3β inhibitor be brought into contact with a stem cells to inhibit GSK3β.

It is envisaged that in performing the present method, GSK3β inhibition will be maintained for a time sufficient to promote differentiation of stem cells into skeletal muscle cells precursors and/or myoblasts and myocytes as the context requires. In one example, the stem cells are contacted with the GSK3β inhibitor for about 4-6 days, or at least about 4 days, or at least about 3 days, or at least about 2 days, or at least about 1 day. In another example, the stem cells are contacted with the GSK3β inhibitor for about 4 days, wherein day 0 represents the day of addition of GSK3β inhibitor. In another example, the concentration of GSK3β inhibitor is about 3 μM. In another example, the concentration of GSK3β inhibitor is up to 3 μM.

It is envisaged that contacting stem cells with a GSK3β inhibitor alone provides conditions sufficient to promote differentiation of stem cells to skeletal muscle cells precursors and/or myoblasts and myocytes. However, it is also envisaged that stem cells can be contacted with other factors that improve the efficiency of the claimed method in that further skeletal muscle cells are produced. It is envisaged that these factors need not explicitly promote differentiation of stem cells to skeletal muscle cells precursors and/or skeletal muscle cells. For example, such additional factors may increase the number of skeletal muscle cells precursors and/or myoblasts and myocytes produced by promoting expansion of the differentiated (e.g. skeletal muscle myoblasts) or differentiating (e.g. skeletal muscle precursor cells) cell populations. Fibroblast Growth Factor (FGF) molecules have been described as potent inducers of mitogenic activity in both embryonic skeletal muscle precursors and adult satellite cells (typically identified by expression of PAX 7).

It is envisaged that the stem cells may be contacted with FGF after contact with the GSK3β inhibitor. However, the stem cells may be contacted with FGF at about the same time as they are contacted GSK3β inhibitor. In an example, the stem cells are contacted with FGF on or about 4 days after contact with the GSK3β inhibitor. In another Example, stem cells are contacted with FGF for about 12-16 days, or at least about 13-15 days. In an example, the stem cells are contacted with FGF for at least about 14 days.

In another example, the method comprises the steps of i) contacting the stem cells with the GSK3β inhibitor; followed by ii) contacting the stem cells with a fibroblast growth factor (FGF). In a particular example, the GSK3β inhibitor is removed prior to the addition of FGF. In another example, the FGF is FGF-2. In a still further example, the concentration of FGF-2 is about 20 ng/ml. In another example, the stem cells are contacted with FGF, in particular FGF-2 for about 14 days (corresponding to days 4 to 18, wherein day 4 represents addition of FGF-2). In another example, the FGF-2 is withdrawn after about 14 days of contact with the stem cells.

In a further example, the stem cells are contacted with the WNT agonist and FGF and no other factors which promote induction and/or differentiation of the cells.

### Purification of Cells

In one example, skeletal muscle cell precursors are purified from a sample of differentiated stem cell progeny. In another example, skeletal muscle cells are purified from a sample of differentiated stem cell progeny. In one example, myocytes and/or myoblasts are purified from a sample. In an example, the sample may be derived from a tissue sample containing pluripotent stem cells. Thus, the method of purifying skeletal muscle cells might also include the harvesting of a source of cells prior to selection using known techniques. Thus, the tissue will be surgically removed. Cells comprising the source tissue (e.g source of pluripotent stem cells) will then be separated into a so called single cell suspension. This separation may be achieved by physical and or enzymatic means.

In another example, the sample is a cell sample comprising pluripotent stem cells. In a further example, sample comprises a mixed population of cells including pluripotent stem cells. By “mixed population of cells” it is meant that the cells may be comprised of cell types that are differentiating, have differentiated or are capable of being differentiated down the skeletal muscle cell lineage. In other examples, the mixed population of cells is a population of cells comprising skeletal muscle precursor cells and/or skeletal muscle myoblasts. In certain examples it may be first necessary to culture the mixed population of cells in a suitable medium to induce cell differentiation down the skeletal muscle cell pathway according to methods as described herein. For example, by inhibiting GSK3β in pluripotent stem cells, then maintaining the cells in culture under conditions sufficient to induce differentiation of the cells into skeletal muscle precursor cells.

The terms “purify”, “purified”, “purifying” or variations thereof are used herein to describe a population of cells in which the proportion, or percentage of cells of one particular cell type or the proportion or percentage of a number of particular cell types is increased when compared with a separate population of the cells (e.g. cells that are HNK-1+).

In one example, the term “purified” is taken to mean that the proportion or percentage of skeletal muscle precursor cells and/or skeletal muscle cells is greater than the proportion or percentage of other cells in the population of cells from which it was originally contained.

The term “population of cells purified for skeletal muscle cells” will be taken to provide explicit support for the term “population of cells comprising X % skeletal muscle cells, wherein X is a percentage as recited herein.

In one example, the population of cells is purified from a mixed population of cells (e.g. differentiated stem cell progeny) comprising skeletal muscle precursor cells and/or myoblasts and myocytes in a selectable form. In this regard, the term “selectable form” will be understood to mean that the cells express a marker (e.g. a cell surface marker) permitting selection of the skeletal muscle precursor cells and/or skeletal muscle myoblasts and myocytes. In a further example, the cells are selected by expression of cell surface markers from C-MET, CXCR4, ACHR and/or HNK1.

Reference to selection of a cell or population thereof does not require selection from a specific tissue source, provided that the tissue comprises cells expressing cell surface markers C-MET, CXCR4, ACHR and/or HNK1.

In another example, the term “purified” is taken to mean that the proportion or percentage of C-Met⁺/CXCR4⁻/HNK-1⁻/ACHR⁻ or C-Met⁺/CXCR4⁺/HNK-1⁻/ACHR⁻ cells is greater than the proportion or percentage of HNK-1+ and/or ACHR⁺ cells in the population of cells from which it was originally contained.

In one example, a population of cells purified for skeletal muscle precursor cells is made up of at least about 15%, at least about 20%, at least about 30%, at least about 40%, at least about 50%, at least about 60% of said cells or at least about 65% of said cells, or at least about 70% of said cells, or at least about 75% of said cells or at least about 80% of said cells, or at least about 85% of said cells, or at least about 87% of said cells, or at least about 90% of said cells, or at least about 95% of said cells, or at least about 96% of said cells, or at least about 97% of said cells, or at least about 98% of said cells, or at least about 99% of said cells, or 100% of said cells.

In one example, a population of cells purified for skeletal muscle cells (e.g. myoblasts and mature myocytes) is made up of at least about 15%, at least about 20%, at least about 30%, at least about 40%, at least about 50%, at least about 60% of said cells or at least about 65% of said cells, or at least about 70% of said cells, or at least about 75% of said cells or at least about 80% of said cells, or at least about 85% of said cells, or at least about 87% of said cells, or at least about 90% of said cells, or at least about 95% of said cells, or at least about 96% of said cells, or at least about 97% of said cells, or at least about 98% of said cells, or at least about 99% of said cells, or 100% of said cells.

The ligands that bind to a cell surface marker according to the present disclosure may be non-antibody based ligands or antibodies or proteins containing an antibody variable region.

Typical non-antibody based ligands include peptides, peptidomimetics, nucleic acid aptamers, peptide aptamers, dendrimers and small organic molecules.

A nucleic acid aptamer (adaptable oligomer) is a nucleic acid molecule that is capable of forming a secondary and/or tertiary structure that provides the ability to bind to a molecular target. An aptamer library is produced, for example, by cloning random oligonucleotides into a vector (or an expression vector in the case of an RNA aptamer), wherein the random sequence is flanked by known sequences that provide the site of binding for PCR primers. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990.

Techniques for synthesizing small organic compounds will vary considerably depending upon the compound, however such methods will be well known to those skilled in the art. In one embodiment, informatics is used to select suitable chemical building blocks from known compounds, for producing a combinatorial library. For example, QSAR (Quantitative Structure Activity Relationship) modelling approach uses linear regressions or regression trees of compound structures to determine suitability. The software of the Chemical Computing Group, Inc. (Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and logP (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, lead compounds identified in initial screens can be used to expand the list of compounds being screened to thereby identify highly active compounds.

Particularly preferred ligands that bind to a cell surface marker are antibodies or antigen binding fragments thereof or proteins comprising an antibody variable region. As used herein the term “antibody” refers to an immunoglobulin molecule capable of binding to a target, such as C-MET, CXCR4, ACHR, HNK-1 and/or an epitope thereof and/or an immunogenic fragment thereof and/or a modified form thereof (e.g., glycosylated) through at least one epitope recognition site, located in the variable region of the immunoglobulin molecule. This term encompasses not only intact polyclonal or monoclonal antibodies, but also variants, fusion proteins comprising an antibody portion with an epitope recognition site of the required specificity, humanized antibodies, human antibodies, chimeric antibodies, and any other modified configuration of the immunoglobulin molecule that comprises an epitope recognition site of the required specificity.

The term “medium” or “media” as used in reference to cell culture, includes the components of the environment surrounding the cells. It is envisaged that the media contributes to and/or provides the conditions sufficient to induce differentiation of the cells into skeletal muscle cells precursors and/or skeletal muscle cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells. In the context of the present disclosure it is preferable that the medium is serum free. In one example, the medium is ITS medium (Dulbecco's modified Eagle's medium F-12 supplemented with insulin, transferrin and selenium).

It will be understood that in performing the methods of the present disclosure, purifying cells carrying any given cell surface marker can be effected by a number of different methods, however, some methods rely upon binding a ligand (e.g., an antibody or antigen binding fragment thereof) to the marker concerned followed by a separation of those that exhibit binding, being either high level binding, or low level binding or no binding. The most convenient ligands are antibodies or antibody-based molecules, such as monoclonal antibodies or based on monoclonal antibodies because of the specificity of these latter agents. Antibodies can be used for both steps, however other agents might also be used, thus ligands for these markers may also be employed to purify for cells carrying them, or lacking them.

In certain examples, the sample may be optionally purified using crude techniques such as drug selection, panning, density gradient centrifugation etc. The particular technique employed will depend upon efficiency of separation, associated cytotoxicity, ease and speed of performance, and necessity for sophisticated equipment and/or technical skill. In another example, a negative selection is performed, where the selection is based on expression of one or more markers found on ES cells, fibroblasts, epithelial cells and the like. Selection may utilise panning methods, magnetic particle selection, particle cell sorter selection, and the like.

Antibodies or ligands may be attached to a solid support to allow for crude separation. Preferably, the separation techniques maximize the retention of viability of the fraction to be collected.

Techniques providing accurate separation include but are not limited to flow cytometry, including magnetic activated cell sorting (MACS) or fluorescence activated cell sorting (FACS). In a preferred example, the sorting utilizes FACS. Methods for performing cell sorting will be apparent to the skilled artisan.

Antibodies against each of the markers described herein are commercially available (see Examples), available from ATCC or other depositary organization and/or can be produced using art recognized techniques.

The disclosure can be practised using cells from a human or any non-human animal species, including but not limited to non-human primate cells, ungulate, canine, feline, lagomorph, rodent, avian and fish cells. Primate cells with which the disclosure may be performed include but are not limited to cells of chimpanzees, baboons, cynomogus monkeys, and any other New or Old World monkeys.

### Uses of the Cells of the Disclosure

The purified skeletal muscle precursor cells and skeletal muscle myoblasts and myocytes of the present disclosure have a variety of uses in clinical therapy, research, development, and commercial purposes.

For example, the cells can be used to prepare a cDNA library. For example, mRNA can be prepared from the cells using standard techniques (e.g. Sambrook et al., A Laboratory Manuel Harbor Laboratory Press 2001; Ausubel et al., Short Protocols in Molecular Biology eds., John Wiley & Sons 1999) and reverse transcribed to produce cDNA. The preparation can then be subtracted with cDNA from undifferentiated stem cells, other precursor cells, or end-stage cells from the skeletal muscle or any other developmental pathway.

The cells of the present disclosure can also be used to generate antibodies that may be specific for additional markers of skeletal muscle precursors and skeletal muscle cells not yet recognised. Polyclonal antibodies can be prepared by injecting vertebrate animal with cells of the present disclosure in an immunogenic form. Production of monoclonal antibodies is described in standard references and U.S. Pat. No. 4, 491,632; U.S. Pat. Nos. 4,472,500 and 4,444,887. Specific antibody molecules can also be produced by contacting a library of immunocompetent cells or viral particles with the target antigen, and growing out positively selected clones. The antibodies in turn can be used to identify or rescue cells of a desired phenotype from a mixed cell population.

The cells of present disclosure are also of interest in identifying expression patterns of transcripts and newly synthesised proteins that are characteristic for skeletal muscle precursors or skeletal muscle myoblasts and myocytes. The expression patterns can be compared with control cell lines, such as undifferentiated pluripotent/embryonic stem cells.

A microarray can be used to analyse gene expression (see Fritz et al., Science 288:316, 2000; www.Gene-Chips.com). An exemplary method is conducted using a Genetic Microsystems array generator, and an AxonGenepix™ Scanner. Microarrays are prepared by first amplifying cDNA fragments encoding marker sequences to be analysed, and spotted directly onto glass slides. To compare mRNA preparations from two cells of interest, one preparation is converted into Cy5-labeled cDA, while the other is converted into Cy3-labeled cDNA. The two cDNA preparations are hybridised simultaneously to the microarray slide, and then washed to eliminate non-specific binding. The slide is then scanned at wavelengths appropriate for each of the labels, the resulting fluorescence is quantified, and the results are formatted to give an indication of the relative abundance of mRNA for each marker on the array.

mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of the test sample of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analysed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample can be performed using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry).

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT WO 95/35505.

In screening for newly synthesised proteins that are characteristic for skeletal muscle precursors or skeletal muscle myoblasts and myocytes, the test sample can be assayed for the level of polypeptide of interest. Polypeptide assay can be accomplished using any of a number of methods to determine the absence or presence or altered amounts of a differentially expressed polypeptide in the test sample. For example, detection can utilize staining of cells or histological sections (e.g., from a biopsy sample) with labelled antibodies, performed in accordance with conventional methods. Cells can be permeabilised to stain cytoplasmic molecules. In general, antibodies that bind a differentially expressed polypeptide of the present disclosure are added to a sample, and incubated for a period of time sufficient to allow binding to the epitope, usually at least about 10 minutes. The antibody can be detectably labelled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

The purified cells of the present disclosure are also useful for in vitro assays and screening to detect factors that are active on cells of the skeletal muscle cell lineage. Of particular interest are screening assays for agents that are active on human cells. A wide variety of assays may be used for this purpose, including immunoassays for protein binding; determination of cell growth, differentiation and functional activity; production of factors; and the like.

The purified cells of the present disclosure may be genetically modified, e.g., to express and/or secrete a protein of interest, e.g., a protein providing a therapeutic and/or prophylactic benefit in skeletal muscle injury or disease. Cells may also be genetically modified to enhance survival, control proliferation and the like. The cells of the present disclosure can also be genetically altered in order to enhance their ability to be involved in tissue regeneration.

Methods for genetically modifying a cell will be apparent to the skilled artisan. For example, a nucleic acid that is to be expressed in a cell is operably-linked to a promoter for inducing expression in the cell. For example, the nucleic acid is linked to a promoter operable in a variety of cells of a subject, such as, for example, a viral promoter, e.g., a CMV promoter (e.g., a CMV-IE promoter) or a SV-40 promoter. Additional suitable promoters are known in the art and shall be taken to apply mutatis mutandis to the present example of the disclosure.

For example, the nucleic acid is provided in the form of an expression construct. As used herein, the term “expression construct” refers to a nucleic acid that has the ability to confer expression on a nucleic acid (e.g. a reporter gene and/or a counter-selectable reporter gene) to which it is operably connected, in a cell. Within the context of the present disclosure, it is to be understood that an expression construct may comprise or be a plasmid, bacteriophage, phagemid, cosmid, virus sub-genomic or genomic fragment, or other nucleic acid capable of maintaining and/or replicating heterologous DNA in an expressible format.

Methods for the construction of a suitable expression construct for performance of the disclosure will be apparent to the skilled artisan and are described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001).

Vectors suitable for such an expression construct are known in the art and/or described herein. The skilled artisan will be aware of additional vectors and sources of such vectors, such as, for example, Life Technologies Corporation, Clontech or Promega.

### Drug Screening

The skeletal muscle precursor cells and/or skeletal muscle myoblasts and myocytes according to the present disclosure can be used to screen for factors (e.g. solvents, small molecule drugs, peptides, oligonucleotides) or environmental conditions (e.g. cell culture) that affect the characteristics of the cells.

One application of the disclosure herein relates to the testing of pharmaceutical compounds for their effect on skeletal muscle tissue maintenance or repair. Screening may be done either because the compound is designed to have a pharmacological effect on the cell, or because a compound designed to have effects elsewhere may have unintended side effects on skeletal muscle cells. The screening is preferably done using the purified CXCR4⁺/C-Met⁺/HNK-1⁻/ACHR⁻ or CXCR4⁻/C-MET⁺/HNK-1⁻/ACHR⁻ or HNK-1⁻/ACHR⁺ cells of the present disclosure.

Assessment of the activity of a candidate pharmaceutical compound generally involves combining the cells of the present disclosure with the candidate compound, either alone or in combination with other drugs. A determination is then made of any change in the morphology, marker phenotype or functional activity of the cells that is attributable to the compound (compared with control untreated cells) and then the effect(s) of the compound correlated with the observed change.

Cytotoxicity can be determined by the effect on cell viability, survival, morphology and the expression of certain markers and receptors. Effects of a compound on chromosomal DNA can be determined by measuring DNA synthesis or repair. An example includes measuring BrdU incorporation. Unwanted effects can also include unusual rates of sister chromatid exchange, determined by metaphase spread.

Effect on cell function can be assessed using any standard assay to observe phenotype or activity of skeletal muscle cells, such as marker expression, receptor binding, contractile activity or electrophysiology. Where an effects is observed, the concentration of the compound can be titrated to determine the median effective dose (ED₅₀).

Agents of interest for screening include known and unknown compounds that encompass numerous chemical classes, primarily organic molecules, which may include organometallic molecules, inorganic molecules, genetic sequences etc. Candidate agents are also found biomolecules, including peptides, polynucleotides, sacchardies, fatty acids, steroids, purine, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants.

### Functional Analysis of Disease Specific iPS Cells

The present disclosure also provides a mechanism for conducting a functional analysis of disease specific induced pluripotent stem (iPS) cells. iPS cells are generally derived by transfection of certain stem-cell associated genes into non-pluripotent cells such as adult fibroblasts. Transfection is achieved through viral vectors bearing the genes Oct-3/4 and Sox2 and may include others that enhance the efficiency of induction. After 3-4 weeks, transfected cells begin to become morphologically and biochemically similar to pluripotent stem cells, and are usually isolated through morphological selection, doubling time, or through a reporter gene and antibiotic selection. Examples of methods to generate iPS cells can be found in Yu J et al., Science 318(5850), 1917-1920 (2007) and Takahashi K et al., Cell 131 (5), 861-872 (2007).

For example, patient-specific human iPS cells can be produced from a subject suffering from a muscular disorder such as muscular dystrophy. Muscular dystrophy encompasses a group of muscle diseases that weaken the musculoskeletal system and hamper locomotion. Muscular dystrophies are characterized by progressive skeletal muscle weakness, defects in muscle proteins, and the death of muscle cells and tissue. One severe type of muscular dystrophy is known as duchenne muscular dystrophy. In this example, the subject specific iPS cells can be coaxed to differentiate into skeletal muscle cells, for example using GSK3β mediated differentiation. The method of the present disclosure can then be applied to purified CXCR4⁺/C-Met⁺/HNK-1⁻/ACHR⁻ or CXCR4⁻/C-MET⁺/HNK-1⁻/ACHR⁻ or HNK-1⁻/ACHR⁺ expressing skeletal muscle cells. The functionality of the cells can then be determined by examining one or more of the following: expression of specific cellular markers indicative of skeletal muscle cells (e.g. PAX3⁺ and PAX7⁺). The cells can also be used to evaluate drugs that may either ameliorate or aggravate the disease phenotype.

Persons skilled in the art would be aware of other skeletal muscle disorders that can be investigated using the above method including muscular dystrophy, hereditary myopathies such as congenital myopathy, distal myopathy and mitochondrial diseases, non-hereditary myopathies such as multiple myositis, dermatomyositis and myasthenia gravis, neurogenic muscular diseases, spinal amyotrophy, bulbar amyotrophy and amyotrophic lateral sclerosis etc.

### Therapeutic Use

The present disclosure also provides for the use of the skeletal muscle precursor cells and/or skeletal muscle myoblasts and myocytes of the invention to enhance tissue maintenance or repair of skeletal muscle in a human patient or other subject in need of such treatment.

To determine the suitability of cell compositions for therapeutic administration, the cells are first tested in an animal model. The cells can be administered to immunodeficient animals. Tissues are harvested after a period of regrowth, and assessed as to whether they are still present. The cells can be labelled with a detectable label e.g. green fluorescent protein. The presence of the administered cells can be assessed by immunohistochemistry or ELISA.

Suitability can be determined by assessing the degree of skeletal muscle recuperation that ensures from treatment of the cells of the invention. For example, muscle necrosis can be induced by injecting cardiotoxin into the tibialis anterior muscles. Injured sites are treated with cell preparations of this disclosure and the muscle tissue examined by histology for the presence of the cells in the damaged area. Muscle function can be monitored by determining such parameters as contractile measurements (e.g. maximum twitch force), fatigue resistance, histological determination of muscle cross sectional area, immunohistochemical or gene expression analysis of markers associated with muscle recovery.

The cells of the present disclosure can be used for tissue reconstitution or regeneration in a human subject or other subject by administering in a manner that permits then to graft or migrate to the intended tissue site and reconstitute or regenerate the functionally deficient area. Special devices are available that are adapted for administering cells capable of reconstituting skeletal muscle function directly to the muscular site of interest. The cells may be administered to a recipient muscle by intramuscular injection.

Subjects which are suitable candidates for treatment according to the methods of the present disclosure include those with muscle damage or injury to be treated is selected from the group comprising direct trauma, laceration, abrasion, bruising, crush injury, contusion injury by short or long impact, tear, strain and/or sprain, muscle injury incurred by cutting or dissection of, or surgical incision into, skeletal muscle, such as muscle injuries that occur during operative or surgical intervention, also known as myotomy, sport injuries, such as torn muscles or torn muscle fibres, and/or injuries occurring from stretching or overstretching muscles and/or injuries incurred during surgery or operation around the spinal cord and/or vertebral column, in addition to rotator cuff ruptures, muscular dystrophy, hereditary myopathies such as congenital myopathy, distal myopathy and mitochondrial diseases, non-hereditary myopathies such as multiple myositis, dermatomyositis and myasthenia gravis, neurogenic muscular diseases, spinal amyotrophy, bulbar amyotrophy and amyotrophic lateral sclerosis etc.

In one example, the purified skeletal muscle cells (skeletal muscle precursor cells and/or myoblasts and myocytes) of the present disclosure are administered to a subject suffering from muscular dystrophy. For example, the injected cells migrate to the injured or diseased muscle. The skeletal muscle cells assemble into muscle tissue resulting in repair or regeneration of the injured or diseased muscle.

Efficacy of treatment can be monitored by measuring improvement in muscle cell function.

Treatment of subjects with purified skeletal muscle cells of the present disclosure according to any of the above described methods may be used in conjunction with other procedures such as surgery.

### Pharmaceutical Compositions

The purified skeletal muscle precursor cells and/or skeletal muscle myoblasts and myocytes of the present disclosure can be supplied in the form of a pharmaceutical composition, comprising a carrier or excipient. The choice of excipient or other elements of the composition can be adapted in accordance with the route and device used for administration.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

The carrier or excipient can be a solvent or dispersion medium containing, for example, water, ethanol, polyol (i.e. glycerol, propylene, glycol and liquid polyethylene glycol and the like), suitable mixtures thereof and/or vegetable oils. The prevention of the action of microorganisms can be brought about by various antibacterial and antifungal agents, for example, parabens, chlorobutanol, phenol, sorbic acid, thimerosal and the like. It may also be preferable to include isotonic agents e.g. sugars or sodium chloride. Stabilising agents can also be added to protect the composition from loss of therapeutic activity. Examples include buffers, amino acids e.g. lysine, carbohydrates such as dextrose, mannose, galactose, fructose, lactose, sucrose, maltose, sorbitol, mannitol etc.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to function as a skeletal muscle cell.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

The composition may also comprises or be accompanied with one or more other ingredients that facilitate the engraftment or functional mobilisation of the cells. Suitable ingredients include matrix proteins or gel polymer that support or promote adhesion of the cells or complementary cell types, especially endothelial cells.

A variety of different scaffolds may be used successfully in the practice of the disclosure. Exemplary scaffolds include, but are not limited to biological, degradable scaffolds. Natural biodegradable scaffolds include collagen, fibronectin, and laminin scaffolds. Suitable synthetic material for a cell transplantation scaffold should be able to support extensive cell growth and cell function. Such scaffolds may also be resorbable. Suitable scaffolds include polyglycolic acid scaffolds, e.g., as described by Vacanti, et al. J. Ped. Surg. 23:3-9 1988; Cima, et al. Biotechnol. Bioeng. 38:145 1991; Vacanti, et al. Plast. Reconstr. Surg. 88:753-9 1991; or synthetic polymers such as polyanhydrides, polyorthoesters, and polylactic acid.

In another example, the cells may be administered in a gel scaffold (such as Gelfoam from Upjohn Company.

The purified skeletal muscle cells precursors and/or skeletal muscle myoblasts and myocytes can be combined with the carrier or excipient in any convenient or practical manner e.g. suspension, emulsification, admixture, encapsulation, absorption and the like.

The compositions described herein may be administered alone or as admixtures with other cells. Cells that may be administered in conjunction with the compositions of the present disclosure include, but are not limited to, other multipotent or pluripotent cells or stem cells, or bone marrow cells. The cells of different types may be admixed with a composition of the disclosure immediately or shortly prior to administration, or they may be co-cultured together for a period of time prior to administration.

The exact amount of cells to be administered is dependent upon a variety of factors, including the age, weight, and sex of the patient, and the extent and severity of the condition to be treated.

In some instances it may be desirable or appropriate to pharmacologically immunosuppress a subject prior to initiating cell therapy and/or reduce an immune response of a subject against the cellular composition. Means for reducing or eliminating an immune response to the transplanted cells are known in the art. As an alternative, the cells may be genetically modified to reduce their immunogenicity.

In another example, the purified cells may be administered with other beneficial drugs or biological molecules (growth factors, trophic factors). When administered with other agents, they may be administered together in a single pharmaceutical compositions, or in separate pharmaceutical compositions, simultaneously or sequentially with other agents (either before or after administration of the other agents).

The present disclosure also provides medical devices for use or when used in a method as described herein according to any example. For example, the present disclosure provides a syringe or catheter or other suitable delivery device comprising purified skeletal muscle cells precursors and/or skeletal muscle myoblasts and myocytes or a composition comprising same according to the present disclosure.

The purified skeletal muscle cells precursors and/or skeletal muscle myoblasts and myocytes or pharmaceutical compositions disclosed herein may be surgically implanted, injected, delivered (e.g. by way of a catheter or syringe), or otherwise administered directly or indirectly to the site in need of repair or augmentation. Exemplary routes of parenteral administration include intravenous, intra-arterial, intramuscular, intraperitoneal, or intrathecal, and infusion techniques.

### Kits

The purified skeletal muscle precursor cells and/or skeletal muscle myoblasts and myocytes or compositions disclosed herein may optionally be packaged in a suitable container with written instructions for a desired purpose, such as the reconstitution of skeletal muscle function to improve some abnormality of the skeletal muscle or for use in screening or diagnostic applications.

The present disclosure also extends to a kit comprising a ligand that binds to C-MET, CXCR4, ACHR and/or HNK-1. The components of the kit may be packaged in aqueous media or in lyophilised form. The kit will typically also contain instructions for use.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

### EXAMPLES

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### Materials and Methods

### Culture of Undifferentiated Cells

Human PSC lines (WA-09 (H9), Mel1, HES3, PDL-iPS) passages p40-65 were maintained on hESC-qualified Matrix (BDMatrigel; BD Biosciences, San Diego, Calif.) in the presence of mTESR1 medium (Stem Cell Technologies, Vancouver BC, Canada), as previously described (Ludwig T and J (2007) Curr Protoc Stem Cell Biol Chapter 1 Unit 1C2).

Directed Differentiation of hPSC into Skeletal Muscle Cells

Experiments were performed using all four hPSC lines. When colony size reached >600 μm in diameter and colony density on the plate was approximately 30-40%, we induced differentiation of hPSC by switching the culture medium from mTESR1 to a chemically defined, serum-free medium DMEM-F12 supplemented with insulin-transferrin-selenium (ITS) (all from Sigma-Aldrich, St. Louis, Mo.). Starting at day 0 of differentiation, cells were cultured in the presence of 3 μM CHIR 99021 (Miltenyi Biotech, Auburn, Calif.) for 4 days. Culture medium was then replaced by ITS containing 20 ng/ml of FGF2 (Miltenyi Biotech) for a further 14 days. For each experimental control condition, hPSCs differentiation was induced as follows: 1) CHIR only: ITS medium containing 3 μM CHIR from days 0-4, followed by ITS medium only until day of analysis; and 2) FGF2 only: ITS medium only between days 0-4, followed by ITS medium containing 20 ng/ml FGF2 for 14 days. Medium was replaced daily until the day of the analysis.

### FACS

Cells were dissociated with 0.05% trypsin or TrypLE Select (Invitrogen, Carlsbad, Calif.) to a single cell suspension and incubated with the appropriate fluorochrome-labelled antibodies (Table 1) at a concentration of 10⁷ cells/ml for 30 minutes on ice. Indirect labelling of HNK and ACHR antibodies was done using goat anti-mouse Alexa Fluor 488 and goat anti-mouse PE (both from Molecular Probes, Invitrogen) as secondary antibodies. Labelled cells were sorted through a BD Influx (five lasers) flow sorter (BD Biosciences) according to the excitation requirements of the fluorochromes. Sorted populations were analyzed using FlowJo software (Tree Star Inc., Ashland, Oreg.).

### Immunohistochemistry

For cytospin preparations of FACS sorted populations, cells were spun onto glass slides using Cytospin 4 (Shandon, Thermofisher, Waltham, Mass.). Cells were then fixed with 100% cold methanol for 5 minutes and subsequently rehydrated in phosphate buffered solution (PBS) for 15-20 minutes. Cultured cells were fixed with 4% paraformaldehyde for 10 minutes at room temperature and permeabilized with 0.3% Triton X-100 in PBS for 30 minutes. A complete list of primary and fluorochrome-labelled secondary antibodies used in this study is provided in the Supporting Information (Table 1). Incubations with primary and subsequently secondary antibodies were performed in Incubation Buffer (0.1% BSA, 2% FBS, 0.1% Triton X-100 in PBS) for 40 minutes at 37° C. Image acquisition was performed on an inverted Nikon Eclipse Ti epifluorescence microscope with the appropriate filter sets using single channel acquisition on a Nikon Digital sight DS-U2 camera. Images were analyzed with Nikon NIS-Elements 3.2 software. All immunofluorescence images are representative of one individual experiment. Experiments were performed n=3 per cell line. Similar results were obtained in all cell lines.

TABLE 1

| | | | | |
|---|---|---|---|---|
| ANTIGEN | FLUOROCHROME | HOST/CLONALITY | DILUTION | COMPANY |

PRIMARY ANTIBODIES

| | | | | |
|---|---|---|---|---|
| Human HGF/C- | APC | mouse IgG1 | 1:50 | R&D Systems |
| MET | | | | |
| CXCR4 | Brilliant Violet 421 | Mouse IgG2a | 1:200 | BioLegend |
| (CD184) | | | | |
| Mab 35 (ACHR) | | Mouse IgG1 | 1:100 | DHSB* |
| Human CD57 | | mouse IgM | 1:200 | Sigma Aldrich |
| (HNK-1) | | | | |
| PAX3 | | mouse IgG2a | 1:100 | R&D Systems |
| PAX7 | | mouse IgG1 | 1:100 | DHSB |
| Myf5 | | rabbit polyclonal | 1:100 | Santa Cruz |
| | | | | Biotechnology |
| Myogenin | | mouse IgG1 | 1:100 | Santa Cruz |
| | | | | Biotechnology |
| MyoD | | rabbit polyclonal | 1:100 | Santa Cruz |
| | | | | Biotechnology |
| MF20 | | mouse IgG2b | 1:100 | DSHB |
| MYHC2 | | mouse IgG2a | 1:100 | DHSB |
| SOX1 | | goat IgG | 1:100 | R&D Systems |
| PARAXIS | | goat IgG | 1:100 | Santa Cruz |
| | | | | Biotechnology |
| Sox10 | | goat polyclonal | 1:100 | R&D Systems |
| AP2a | | mouse IgG2b | 1:100 | DHSB |
| LMX1A | | rabbit polyclonal | 1:200 | Abnova |
| Six4 | | Mouse IgG1 | 1:100 | Abnova |

SECONDARY ANTIBODIES

| | | | | |
|---|---|---|---|---|
| Anti-rabbit IgG | Alexa Fluor 647 | donkey | 1:400 | Molecular Probes (Invitrogen) |
| (H + L) | | | | |
| Anti-goat IgG | Alexa Fluor 647 | donkey | 1:400 | Molecular Probes (Invitrogen) |
| (H + L) | | | | |
| Anti-goat IgG | Alexa Fluor 488 | donkey | 1:400 | Molecular Probes (Invitrogen) |
| (H + L) | | | | |
| Anti-mouse | Alexa Fluor 555 | donkey | 1:400 | Molecular Probes (Invitrogen) |
| IgG2a | | | | |
| Anti-mouse | Alexa Fluor 488 | goat | 1:400 | Molecular Probes (Invitrogen) |
| IgG (H + L) | | | | |
| Anti-mouse | Alexa Fluor 555 | donkey | 1:400 | Molecular Probes (Invitrogen) |
| IgG2b | | | | |
| Anti-mouse | Alexa Fluor 647 | goat | 1:400 | Molecular Probes (Invitrogen) |
| IgG1 | | | | |
| Anti-mouse IgM | Alexa Fluor 555 | goat | 1:400 | Molecular Probes (Invitrogen) |
| Anti-rabbit IgG | Alexa Fluor 488 | goat | 1:400 | Molecular Probes (Invitrogen) |
| (H + L) | | | | |
| Anti-mouse | Alexa Fluor 555 | goat | 1:400 | Molecular Probes (Invitrogen) |
| IgG1 (H + L) | | | | |
| Anti-mouse | PE | goat | 1:400 | Molecular Probes (Invitrogen) |
| IgG1 | | | | |

*(Developmental Hybridoma Studies Bank)

### Gene Expression Analysis

Total RNA was extracted using RNeasy Mini kit (Qiagen, Hilden, Germany), and DNAse I treatment (Qiagen) was performed to avoid genomic DNA contamination. The Ambion RETROscript First Strand Synthesis Kit (Invitrogen) was used to reverse-transcribe total RNA (500 ng each sample). Polymerase chain reaction (PCR) was performed using the Mastercyler proS (Eppendorf AG, Hamburg, Germany). The inventors optimized the PCR conditions and determined the linear amplification range for each primer by varying annealing temperature and cycle number. Primer sequences, cycle numbers and annealing temperatures are provided in Table 2. All RT-PCR data shown are representative of one individual experiment. Experiments were performed n=3 per cell line. Similar results were obtained in all cell lines.

TABLE 2

| | | | | AMPLI- |
|---|---|---|---|---|
| | | | ANNEAL- | CON |
| | | SEQUENCE | ING | LENGTH |
| Gene | Orientation | 5′ to 3′ | TEMP °C. | bp |
| LBX1* | forward | AAAGTCGCGCACG | 59 | 249 |
| | | GCCTTCA | | |
| | | (SEQ ID NO: 1) | | |
| | reverse | GCCAGCGCCACGA | | |
| | | TGTCCAT | | |
| | | (SEQ ID NO: 2) | | |
| PAX7* | forward | ACCCCTGCCTAA | 60 | 121 |
| | | CCACATC | | |
| | | (SEQ ID NO: 3) | | |
| | reverse | GCGGCAAAGAATCT | | |
| | | TGGAGAC | | |
| | | (SEQ ID NO: 4) | | |
| PAX3* | forward | TACAGGTCTGGTT | 57 | 183 |
| | | TAGCAAC | | |
| | | (SEQ ID NO: 5) | | |
| | reverse | GATCTGACACAGC | | |
| | | TTGTGGA | | |
| | | (SEQ ID NO: 6) | | |
| MYF5 | forward | TTCTCCCCATCCC | 59 | 235 |
| | | TCTCGCT | | |
| | | (SEQ ID NO: 7) | | |
| | reverse | AGCCTGGTTGACCT | | |
| | | TCTTCAG | | |
| | | (SEQ ID NO: 8) | | |
| MYH2 | forward | CCGCCCTTGACAA | 55 | 220 |
| | | AAAGCAA | | |
| | | (SEQ ID NO: 9) | | |
| | reverse | GCGCAGGATCTTT | | |
| | | CCCTCTT | | |
| | | (SEQ ID NO: 10) | | |
| ACHR | forward | GCTAACCCTCACCA | 59 | 346 |
| | | ACCTCAT | | |
| | | (SEQ ID NO: 11) | | |
| | reverse | GGTTGCTGCACT | | |
| | | TTGGTCC | | |
| | | (SEQ ID NO: 12) | | |
| MYOD* | forward | GCGCGCTCCTGAA | 60 | 166 |
| | | ACCCGAA | | |
| | | (SEQ ID NO: 13) | | |
| | reverse | TCGGCGTTGGTGG | | |
| | | TCTTGCG | | |
| | | (SEQ ID NO: 14) | | |
| TBX6* | forward | TTCCCGGCTCTCA | 60 | 143 |
| | | CCTCCGT | | |
| | | (SEQ ID NO: 15) | | |
| | reverse | TGGCCTGCACCAG | | |
| | | TGTGTGT | | |
| | | (SEQ ID NO: 16) | | |
| MESP1* | forward | CACACCTCGGGCTC | 60 | 119 |
| | | GGCATAAA | | |
| | | (SEQ ID NO: 17) | | |
| | reverse | CAGGCCGCAGAGAG | | |
| | | CATCCAG | | |
| | | (SEQ ID NO: 18) | | |
| GAPDH* | forward | CCCCTTCATTGACC | 60 | 342 |
| | | TCAACTACA | | |
| | | (SEQ ID NO: 19) | | |
| | reverse | TTGCTGATGATCTTG | | |
| | | AGGCTGT | | |
| | | (SEQ ID NO: 20) | | |
| SIX4* | forward | CCATGCTGCTGGCT | 60 | 164 |
| | | GTGGGAT | | |
| | | (SEQ ID NO: 21) | | |
| | reverse | AGCAGTACAACACAG | | |
| | | GTGCTCTTGC | | |
| | | (SEQ ID NO: 22) | | |
| PARAXIS* | forward | AGGGCCACGGAGA | 61 | 120 |
| | | TGAGCCT | | |
| | | (SEQ ID NO: 23) | | |
| | reverse | GGTCCCCCGGTCC | | |
| | | CTACACA | | |
| | | (SEQ ID NO: 24) | | |
| SIX1* | forward | GTCCAGAACCTCCC | 50 | 101 |
| | | CTACTCC | | |
| | | (SEQ ID NO: 25) | | |
| | reverse | CGAAAACCGGAGTC | | |
| | | GGAACTT | | |
| | | (SEQ ID NO: 26) | | |
| SOX10 | forward | CCCACACTACACC | 59 | 143 |
| | | GACCAG | | |
| | | (SEQ ID NO: 27) | | |
| | reverse | GGCCATAATAGGGT | | |
| | | CCTGAGG | | |
| | | (SEQ ID NO: 28) | | |
| MSGN1* | forward | GGAGGCGGAAAGCC | 60 | 193 |
| | | AGCGAGA | | |
| | | (SEQ ID NO: 29) | | |
| | reverse | CTGGGCTCTCTGCC | | |
| | | GCGGTTA | | |
| | | (SEQ ID NO: 30) | | |
| T* | forward | CGATCCTGGGTG | 55 | 220 |
| | | TGCGTAA | | |
| | | (SEQ ID NO: 31) | | |
| | reverse | GACCAAGACTGT | | |
| | | CCCCGCT | | |
| | | (SEQ ID NO: 32) | | |
| SOX1 | forward | GAGCTGCAACTTGG | 60 | 271 |
| | | CCACGAC | | |
| | | (SEQ ID NO: 33) | | |
| | reverse | GAGACGGAGAGGAA | | |
| | | TTCAGAC | | |
| | | (SEQ ID NO: 34) | | |
| AP2a | forward | AGGCAGAGCCAGGA | 60 | 464 |
| | | GTCTGGGCT | | |
| | | (SEQ ID NO: 35) | | |
| | reverse | CGGAGCACTCCGCC | | |
| | | CAGCAGCGA | | |
| | | (SEQ ID NO: 36) | | |
| LMX1A | forward | TCCTAGCCTTGGAGA | 59 | 271 |
| | | AGCAACT | | |
| | | (SEQ ID NO: 37) | | |
| | reverse | CAGTGACTGGAGCA | | |
| | | GAGAGAA | | |
| | | (SEQ ID NO: 38) | | |

*Primers used for Quantitative PCR

For quantitative PCR, GAPDH was used as a reference gene and reactions were run using LightCycler480 SYBR Green I Master (Roche Applied Science, Indianapolis, Ind.) on a LightCycler 480 system (Roche Applied Science). Target gene expression was normalized to the reference gene (GAPDH), and subsequent quantification of gene expression was compared relative to day 0 undifferentiated hPSCs (Pfaffl, (2001) Nucleic acids Res 29, e45).

### Culture of FACS Isolated Cell Populations

FACS purified ACHR+ myocytes, CXCR4−/C-MET+ and CXCR4+/C-MET+ precursors were plated onto tissue culture wells coated with 2 μg/ml fibronectin and 2 μg/ml laminin (both from Invitrogen) in ITS medium supplemented with 10 μM Rock Inhibitor Y-27632 (Sigma Aldrich). Myocytes were maintained in ITS medium in the presence of 50 ng/ml IGF1 (Peprotech, Rocky Hill, N.J.) until analyzed. Precursor cell populations were cultured in ITS medium until terminal muscle differentiation.

### Statistical Analysis

Data were analyzed by two-way analysis of variance (ANOVA) followed by Bonferroni's post-test to calculate p values. Analyses were performed using statistical software (GraphPad Prism 5.04; GraphPad Software, San Diego, Calif.). Probability values <0.05 were considered statistically significant. Error bars in each figure represent the standard error of the mean of three or more individual experiments. For qPCR data, p values were calculated for changes in expression of markers over time compared to day 0. For quantitative analysis of FACS sorting data, the percentage of myogenic cells relative to the total number of cells were obtained for each experimental culture treatment, and p values were calculated for differences between the means of each experimental condition.

### Example 1

### Derivation of Skeletal Muscle Cells from hPSCs

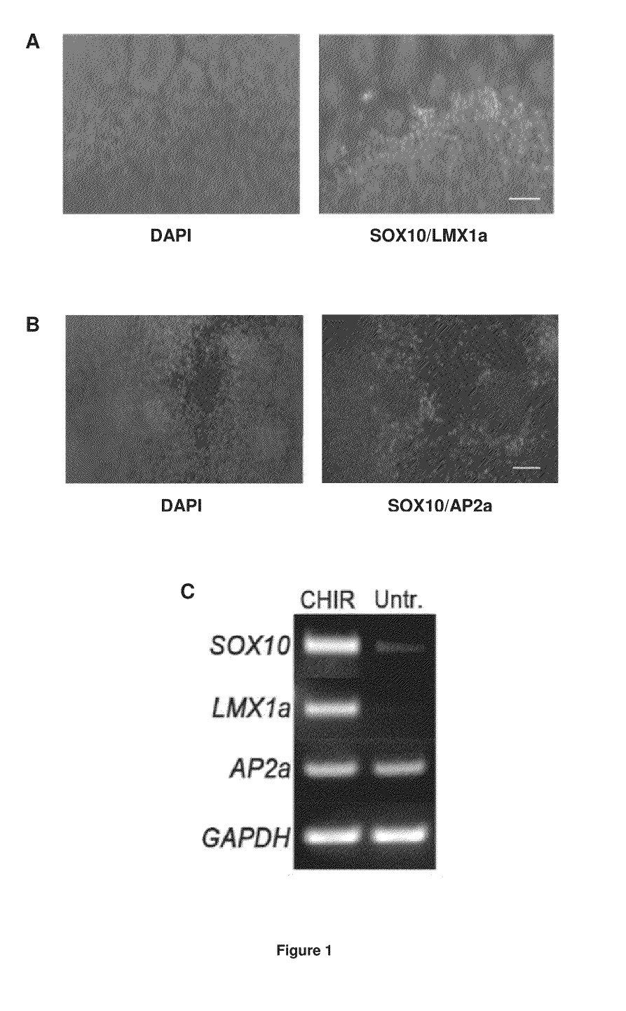

Differentiation of hPSCs was initiated at medium to large colony size (diameter 600 μm) and low colony density, in serum-free medium consisting of Dulbecco's modified Eagle's medium F-12 (DMEM-F12) supplemented with insulin, transferrin and selenium. Paraxial mesoderm specification of hPSCs was achieved through the activation of WNT/beta-catenin signaling mediated by the small molecule GSK-3β inhibitor CHIR 99021 (Cohen P et al. (2004) Nat Rev Drug Disc 3:479-487, Tan J Y et al (2013) Stem Cell Dev 22:1893-1906). GSK-3β is known to target a number of substrates for phosphorylation, one of which is beta-catenin, an integral transducer within the canonical WNT signaling pathway. Therefore, inhibition of GSK-3β activity prevents the targeted phosphorylation of beta-catenin, rendering it resistant to degradation thus leading to activation of TCF-mediated transcription of downstream target genes (Wu D and Pan W (2010) Trends Biochem Sci 35:161-168). In addition to paraxial mesoderm, WNT signaling is a potent inducer of dorsal cell fates such as roof plate, neural crest and non-neural ectoderm, marked by LMX1A, SOX10 and AP2α, respectively (Gammill and Bronner-Fraser (2003) Nat Rev Neurosci 4:795-805; Millonig et al (2000) Nature 403:764-769) (FIG. 1).

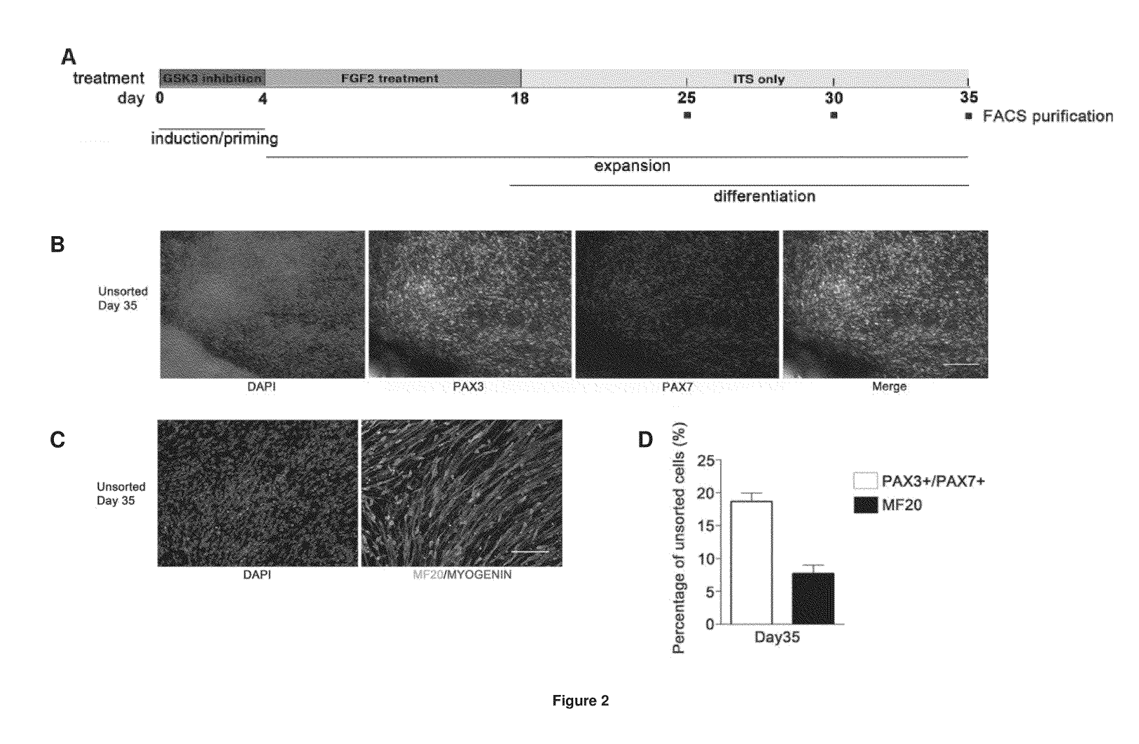

Human PSCs were first exposed to 3 μM CHIR for 4 days, and then the small molecule was replaced with 20 ng/ml of FGF2 for an additional 2 weeks (FIG. 2A). To optimize the differentiation of hPSCs towards a myogenic phenotype, different CHIR concentrations were tested. High toxicity was found at >3 μM and inefficient induction at doses of <3 μM. The FGF signaling pathway has been identified to regulate several developmental processes of muscle formation. During somitogenesis, segmentation determination is mediated by an FGF signaling gradient within the presomitic mesoderm (Aulehla and Pourquie (2010) Cold Spring Harb. Perspect. Biol. 2, a000869. The addition of FGF2 was to drive expansion of the muscle precursor compartment within the culture system.

Following withdrawal of FGF2 and a further 17 days of culture in ITS medium alone areas with skeletal muscle cells were scored in treated culture dishes, prior to FACS analysis and identified by immunocytochemistry as PAX3+ and PAX7+ precursors (FIG. 2B) (Relaix et al (2005) Nature 435:948-953) and bipolar skeletal myocytes, positive for myogenin and sarcomeric myosin (MF20) (FIG. 2C). Quantitative analysis revealed the percentage of total PAX3^{+/}PAX7⁺ and MF20⁺ muscle cells within the cell culture to be >18% and >8%, respectively, demonstrating the robustness of the treatment strategy.

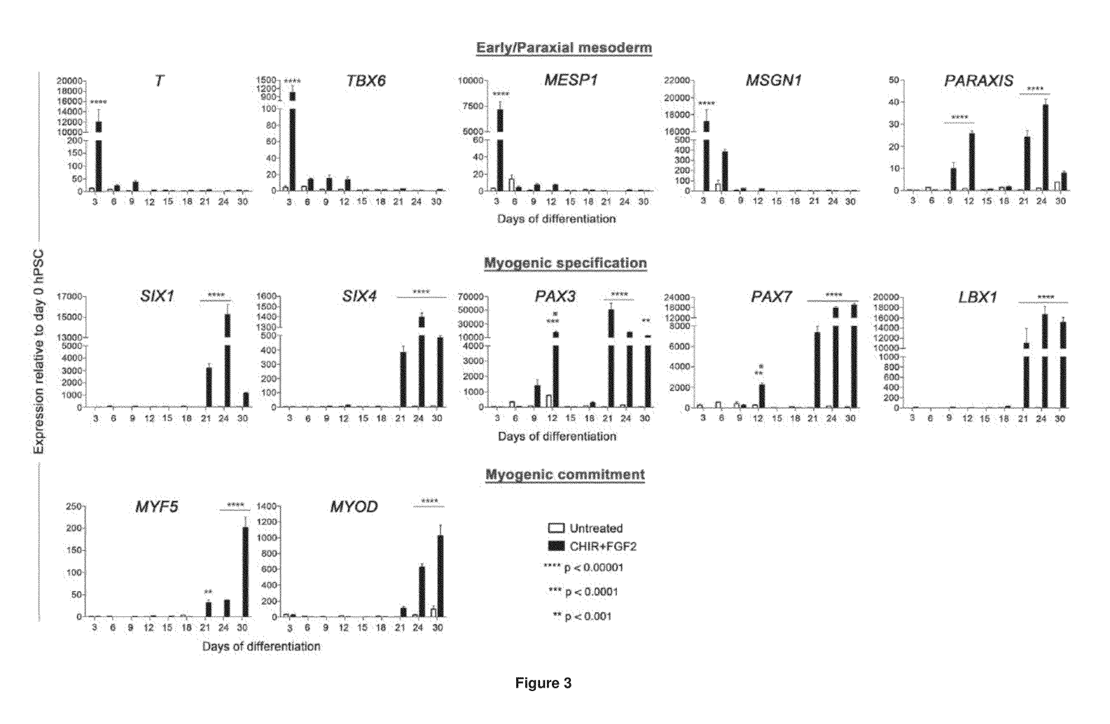

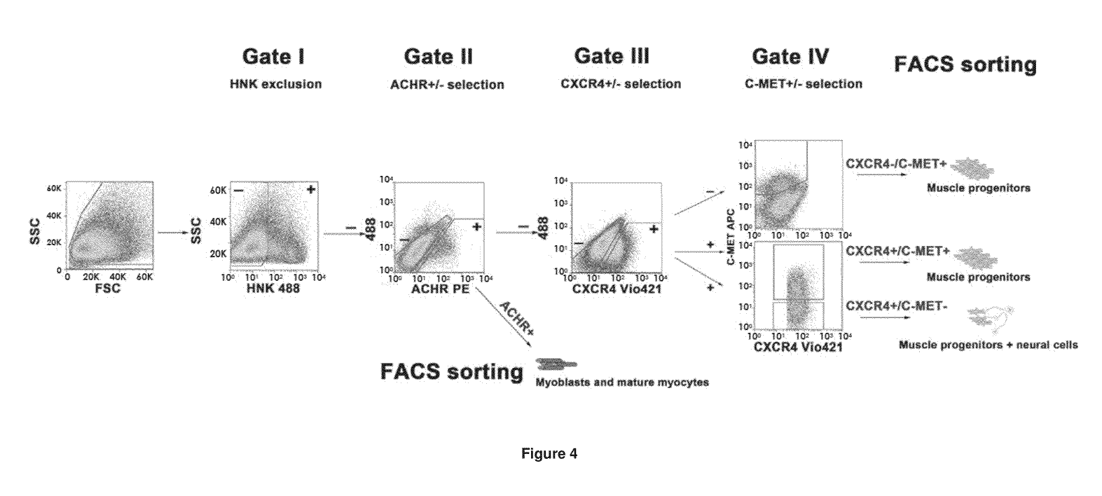

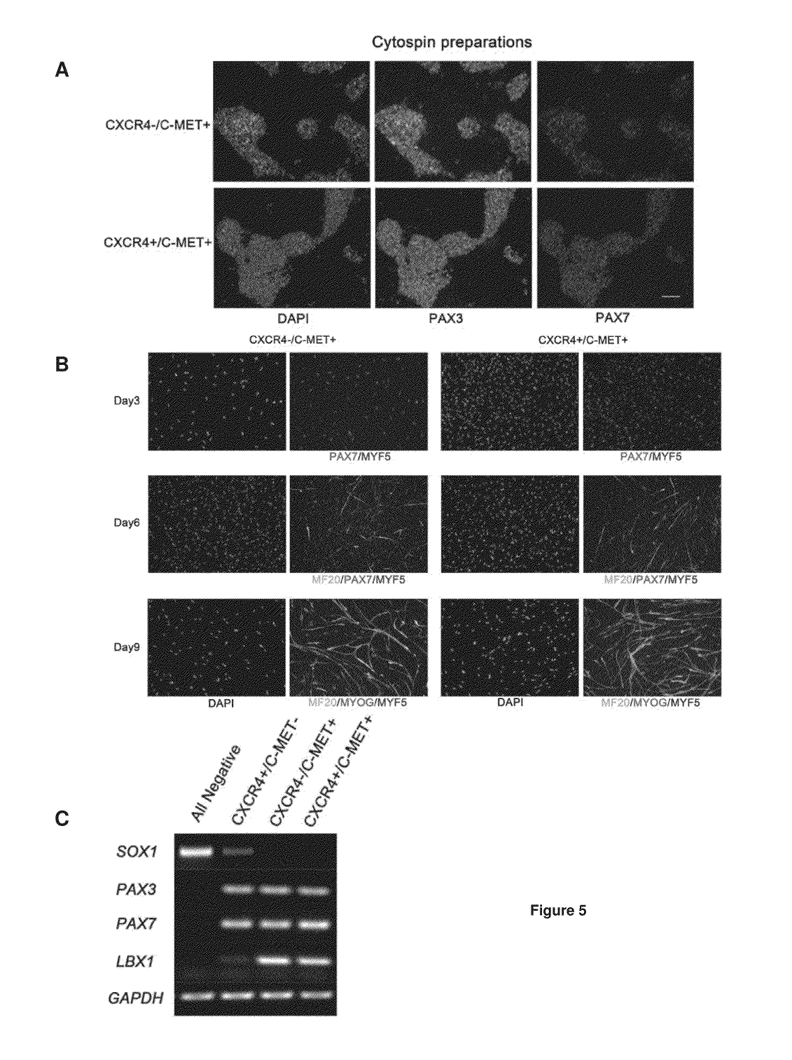

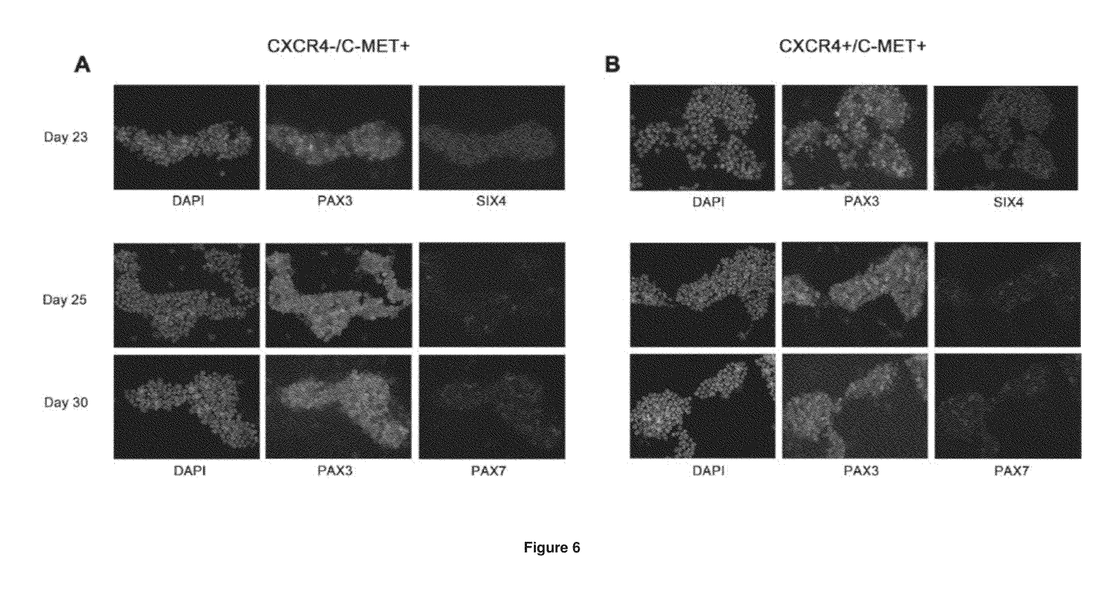

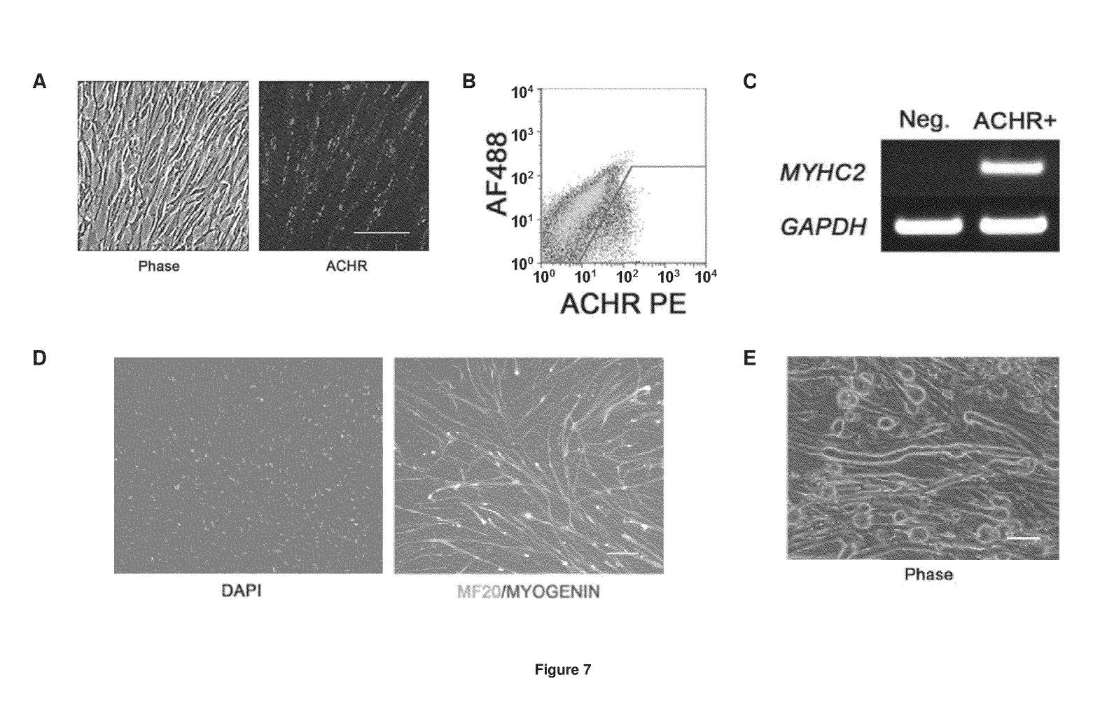

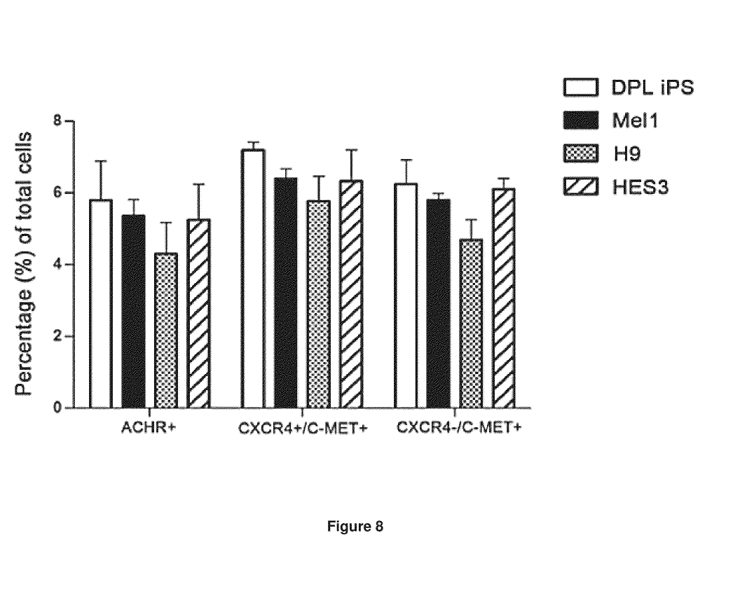

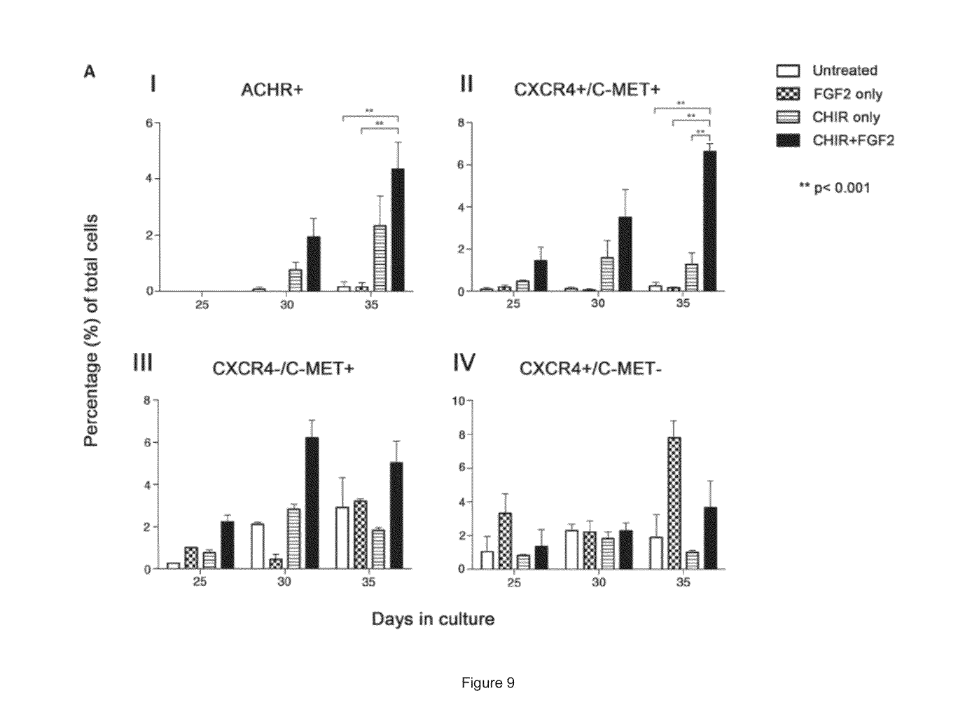

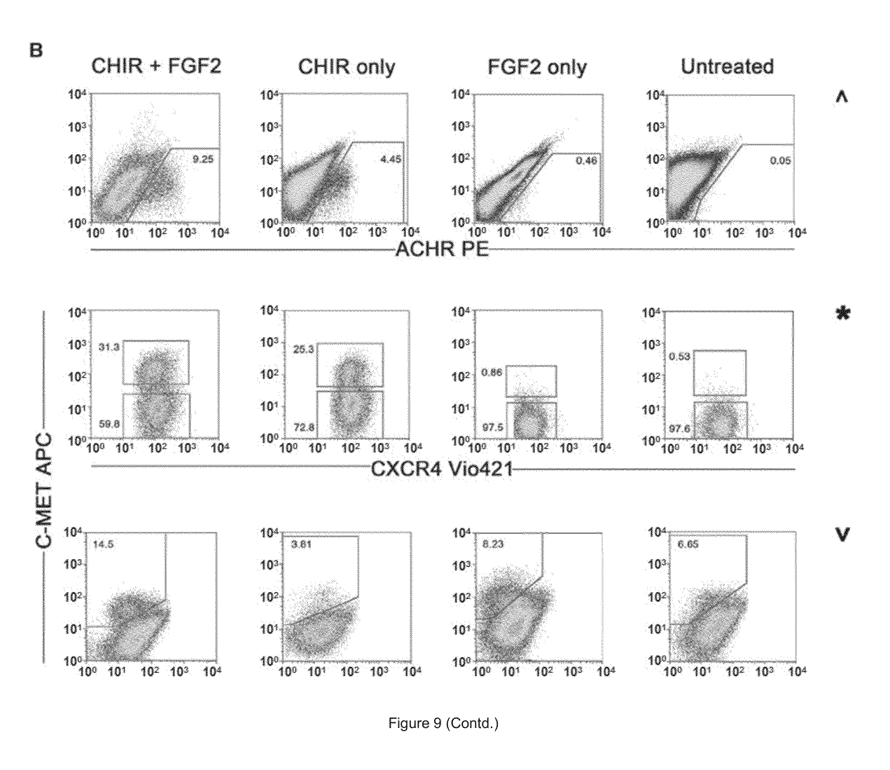

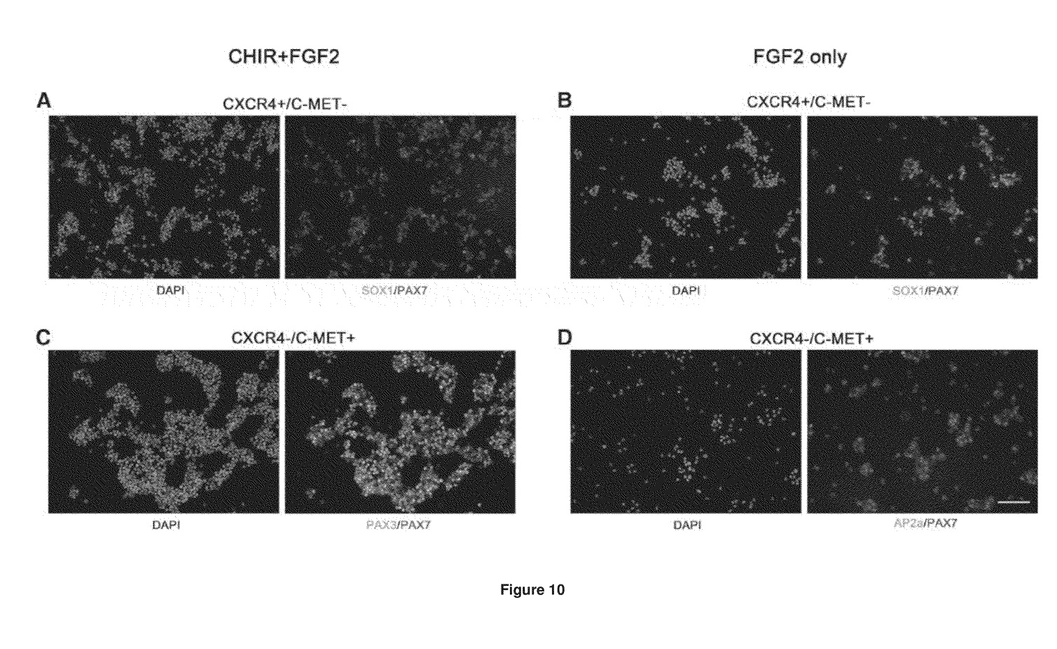

To further profile the efficacy of the treatment, the inventors analyzed the expression of key regulatory genes associated with the acquisition of a myogenic cell fate by quantitative PCR (qPCR). Data were acquired during a fixed 3-day interval starting at day 0 and ending at day 30 of in vitro differentiation in CHIR+FGF2 compared to untreated hPCSs (FIG. 3). Expression profiling of differentiating hPSCs over the course of treatment, showed the guided progression of hPSCs through key myogenic milestones. Inhibition of GSK3β resulted in a marked increase in the expression of paraxial/presomitic mesoderm genes such as TBX6, Mesogenin (MSGN1) and MESP1) with an early peak at day 3 of differentiation. Subsequent PARAXIS activation starting at day 9 of differentiation indicated progression towards somitic mesoderm. Significantly, expression of the muscle specification genes SIX1 and SIX4, PAX3, PAX7 and the migratory muscle precursor marker LBX1 exhibited marked activation at day 21 of differentiation under treatment conditions. Expression of the myogenic regulatory factors MYF5 and MYOD indicated muscle commitment and progression of the myogenic differentiation program (Rudnicki et al., (1993) Cell 75:1351-1359). In contrast, an insignificant activation of myogenic-specifier genes occurred during differentiation of untreated hPSCs.

Interestingly, Paraxis exhibited a second peak of expression beginning at day 21, correlating with the activation of SIX1, SIX4, PAX3 and PAX7. While known to regulate somite epithelization, Paraxis has also been shown to be expressed in migratory hypaxial muscle precursors. Therefore, secondary activation of PARAXIS expression, in conjunction with expression of LBX1, suggests a bias towards hypaxial myogenesis within this system.

### Example 2

### FACS Isolation of Hypaxial Skeletal Muscle Precursors

The expression of the migratory skeletal muscle precursor marker LBX1 observed in CHIR treated differentiating hPSCs lead the inventors to purify this putative migratory muscle compartment by using CXCR4 and C-MET surface markers, which together are reported to define migratory muscle precursor. Within the hypaxial domain of the embryonic dermomyotome, C-MET expression is critical for the delamination of PAX3+ LBX1+ migratory muscle precursors, while subsequent survival and distribution of precursors at the site of migration is CXCR4 dependent. However, CXCR4 and C-MET may also be expressed in cells of different origins such as neural and neural crest cells, respectively. Therefore, to exclude these cell types, CD57/HNK-1 was used as a negative selection marker. Although, CXCR4 can be used to define and isolate definitive endoderm from hPSCs, CHIR treatment used in the current protocol was not permissive for the generation of endoderm cells, as confirmed by the lack of specific endodermal markers in the cultures. Due to the observed activation of myogenic specification genes beginning at day 21 of hPSC differentiation (FIG. 3), skeletal muscle precursors were isolated by FACS at three time-points, day 25, 30 and 35. The isolated cell populations were: HNK−/ACHR−/CXCR4−/C-MET− (all negative), HNK−/ACHR−/CXCR4+/C-MET− (CXCR4+/C-MET−); HNK−/ACHR−/CXCR4+/C-MET+ (CXCR4+/C-MET+) and HNK−/ACHR−/CXCR4−/C-MET+ (CXCR4−/C-MET+). A detailed gating strategy used for the FACS protocol is shown in FIG. 4.

Post-sorting analysis revealed the presence of myogenic cells only in the populations where CXCR4 and/or C-MET were present (CXCR4+/C-MET−, CXCR4+/C-MET+ and CXCR4−/C-MET+) FIG. 5. To quantify the level of purity in these populations, an immediate post-sort immunocytochemical analysis was performed on cytospin preparations. The cytocentrifugation technique spins a cell suspension onto a defined area of a glass slide creating a monolayer of flattened cells, allowing prominent nuclear presentation. Based on nuclear staining, only CXCR4−/C-MET+ and CXCR4+/C-MET+ cell populations allowed the isolation of highly pure skeletal muscle precursors.

At day 35, the percentage of total cells immunoreactive for the muscle stem cell marker PAX3 was 97±0.5% in CXCR4−/C-MET+ and 98±0.2% in CXCR4+/C-MET+. The percentage of PAX7 was 84±1.7% in CXCR4−/C-MET+ and 96±2.8 in CXCR4+/C-MET+ (FIG. 5A). Immunocytochemical analysis of precursor populations sorted at earlier time-points revealed developmental progression of the myogenic program. CXCR4−/C-MET+ and CXCR4+/C-MET+ cells sorted at day 23 were characterized by expression of early myogenic specifier genes SIX4 and PAX3 prior to PAX7 expression. Subsequent acquisition of PAX7 expression, starting at day 25, marked lineage progression (FIG. 6). By day 35, close to all CXCR4−/C-MET+ CXCR4+/C-MET+ cells co-expressed PAX3 and PAX7 (FIG. 5A). However, an overall lower expression of PAX7 was observed in CXCR4−/C-MET+ cells compared to CXCR4+/C-MET+ cells. Due to the earlier activation of Pax3 (Horst et al (2006) Int J Dev Biol 50:47-54) and the expression of Cxcr4 in late-stage migratory precursors (Vasyutina et al., (2005) Genes Dev 19:2187-2198) during muscle development, it was speculated that CXCR4−/C-MET+ cells could represent a more primitive precursor/progenitor population.

Post-sorting cultures of CXCR4−/C-MET+ and CXCR4+/C-MET+ cells isolated at day 35 of hPSC differentiation confirmed the validity of the sorting strategy, with all plated cells from both populations undergoing progressive terminal muscle differentiation as shown by expression of MYF5, MYOG and MF20 (FIG. 5B). After 3 days of culture few cells retained expression of PAX7 while all cells expressed MYF5 indicating muscle commitment. By day 9 the majority of cells were in an advanced stage of muscle differentiation.

Gene expression analysis by RT-PCR confirmed the immunocytochemical data, demonstrating the presence of PAX3 and PAX7 mRNA transcripts together with LBX1 in both CXCR4−/C-MET+ and CXCR4+/C-MET+ sorted populations and, importantly, their absence in all negative cell population (FIG. 5C).

Although enriched in muscle precursors, the CXCR4+/C-MET− cell population showed heterogeneity, with gene expression analysis revealing the presence of muscle together with SOX1+ neural cells (FIG. 5C) and thus would not be useful for in vitro or in vivo studies.

### Example 3

### Single Step Isolation of Mature Skeletal Myocytes

Besides their potential use in clinical applications, enriched populations of hPSC-derived skeletal muscle cells also provide a platform for basic research investigations. Purification of hPSC-derived mature myocytes offers an unlimited source of cells for large scale screening of novel therapeutic compounds and toxicity studies. To this end, a simple single-antigen strategy was set up for the direct isolation and purification of mature skeletal myocytes.

In the two-step culture system, bipolar skeletal myocytes appeared at approximately 4 weeks of hPSC differentiation. Immunocytochemical analysis revealed strong expression of the muscle-specific nicotinic acetylcholine receptor (ACHR) on these cells (FIG. 7A). This family of receptors are among the first membrane proteins to be expressed during skeletal muscle development. However, their presence is required only later during synaptogenesis when they mediate synaptic transmission at the neuromuscular junction. At days 30 and 35 of hPSC differentiation, an easily distinguishable ACHR+ population (up to 8% of total cells) was identified and isolated by FACS (FIG. 7B). Analysis of both ACHR+ and ACHR-fractions showed that expression of the mature muscle marker myosin heavy chain 2a (MYHC2) was restricted only to ACHR+ cells (FIG. 7C).

Following isolation, the ACHR+ cells were plated onto fibronectin/laminin coated plates in the presence of ITS medium. At 24 hours after plating, as expected, all ACHR+ cells were immunoreactive for the mature muscle markers myogenin and MF20 (FIG. 7D). Prolonged cell culture (>20 days) of ACHR+ cells led to the progressive fusion of myocytes into multinucleated myotubes (FIG. 7E).

### Example 4

### GSK-3 Inhibition is Required for Efficient Muscle Derivation

The efficacy of the two-step protocol was determined by comparing the following sorted populations: ACHR+; CXCR4+/C-MET+; CXCR4−/C-MET+ and CXCR4+/C-MET− derived under different culture conditions. The conditions were: CHIR+FGF2, CHIR only, FGF2 only, and untreated. Muscle precursors were already present at day 25 of hPSC differentiation as indicated by the presence of both CXCR4+/C-MET+ (II) and CXCR4−/C-MET+ (III) cell populations. As expected, the overall percentage of each myogenic population increased overtime and thus at day 35, under CHIR+FGF2 treatment the inventors obtained collectively from ACHR+, CXCR4+/C-MET+ and CXCR4−/C-MET+ cell populations, a total of up to 20% of muscle cells (FIG. 6A I, II, III). Significantly, a large component of these cells were PAX3+ and PAX7+ precursors (CXCR4+/C-MET+; CXCR4−/C-MET+) comprising of more than 12% of total cells (FIG. 6A II, III). A similar robust percentage of muscle cells was observed across all four cell lines (3 hESC and 1 hiPS), demonstrating the efficiency of the two-step protocol (FIG. 8).

CHIR+FGF2 treatment resulted in the efficient derivation of muscle precursors, however, exposure of hPSCs to CHIR only was sufficient for myogenic induction. Reduction in the percentage of CXCR4+/C-MET+ and ACHR+ populations compared to CHIR+FGF2 cultures indicated an active role for FGF2 in the expansion of the myogenic compartment (FIGS. 9A I, II 6B top and middle). In stark contrast, the absence of CHIR treatment resulted in almost complete loss of both of these cell fractions (FIGS. 9A I, II, 6B top, middle). Interestingly, the overall percentage of CXCR4+/C-MET− and CXCR4−/C-MET+ cells did not change significantly among the four different treatment conditions (FIGS. 9A III, IV, 6B middle, bottom). However, comparative analysis of cell composition between these cell fractions isolated from CHIR+FGF2 or FGF2 only treated cultures revealed a fundamental shift from a myogenic to a non-myogenic cell fate in the absence of CHIR treatment (FIG. 10). These data illustrate a requirement of CHIR-mediated GSK3β inhibition for the robust induction of muscle cells from hPSCs. The addition of FGF2 is then necessary to achieve optimal expansion of skeletal muscle precursors.

### Remarks

A simple two-step differentiation method is presented that recapitulates the early events of embryogenesis to efficiently derive PAX3+/PAX7+ skeletal muscle precursors from hPSCs. We demonstrate the feasibility of deriving robust numbers of skeletal muscle cells without the aid of transgene driven differentiation. Central to this method was the activation of canonical WNT signaling by the GSK3β inhibitor CHIR. Expression profiling of hPSCs over the course of guided differentiation showed progression through defined developmental milestones leading to myogenesis. This transition was initiated by a strong induction of TBX6, MESP1 and MSGN1 in CHIR-treated hPSCs, followed by high levels of PARAXIS expression, indicating progression into somitic mesoderm. In addition to the induction of paraxial mesoderm, activation of WNT signaling by CHIR was responsible for the generation of dorsal tissues, such as dorsal neural tube cells marked by LMX1A expression, along with SOX10+ neural crest cells and AP2α+ non-neural ectoderm (FIG. 1). It has been established that myogenic patterning of the dermomyotome requires WNT signaling from the dorsal neural tube and overlying ectoderm, together with transient, neural crest-mediated notch activation of myogenic precursors. Early GSK3β inhibition during hPSC differentiation allowed reproduction of the conditions necessary for the specification of skeletal muscle cells, closely replicating the events that occur during normal development in vivo. While not wishing to be bound by theory, the inventors speculate that the generation of dorsal tissues played an essential role in delivering the appropriate signals required for the patterning of the pre-somitic mesoderm within our culture system. Conversely, prolonged exposure to CHIR for up to 10 days demonstrated to have a negative effect on muscle derivation and no muscle cells were identified in the treated dishes. Although it was shown that CHIR alone is sufficient for myogenic induction, prolonged FGF2 exposure proved to play a proliferative role by significantly increasing the number of myogenic precursors. The robustness of the protocol was validated by obtaining similar results with 4 hPSC lines, confirming that small molecule-mediated GSK3β inhibition is a simple but highly efficient approach to direct differentiation of hPSCs into skeletal muscle precursors.

Progress in considering hPSC-derived muscle as a valid source of cells for basic and translational research applications has been hindered by the lack of an efficient method to isolate muscle precursors. To overcome this limitation a FACS strategy was developed to purify muscle precursors generated in the differentiation system. Due to the detection of LBX1 transcripts during directed myogenic commitment of hPSCs, two markers, C-MET and CXCR4 that are known to be highly expressed in hypaxial migratory muscle precursors during development were considered. FACS selection of two populations, CXCR4−/C-MET+ and CXCR4+/C-MET+, allowed the isolation of PAX3+/PAX7+ precursors at high purity. Notably, the negative cell population (HNK−/ACHR−/CXCR4−/C-MET−) was devoid of any muscle markers indicating not only that the sorting strategy is sufficient to isolate all skeletal muscle cells generated in the culture system, but that all PAX3+/PAX7+ precursors are of hypaxial origin. The specificity of this strategy was also confirmed by the complete absence of CXCR4+/C-MET+ cells and by a non-muscle identity of CXCR4−/C-MET+ cells in the absence of early GSK3β inhibition during hPSC differentiation.

Transplantation of highly purified skeletal muscle precursors has been considered a possible option for the treatment of degenerative muscle disorders, such as muscular dystrophy. These findings will accelerate the evaluation of the therapeutic potential of hPSC-derived muscle cells in preclinical models. Moreover, future application of our method to patient-specific iPS cell lines will help to study muscle development during disease.

Concomitant with the isolation of skeletal muscle precursors, a simple strategy for the direct isolation of mature skeletal myocytes through the positive selection of ACHR+ cells is described. This highly efficient derivation and direct isolation of mature embryonic stage skeletal myocytes provides an unprecedented platform for developmental modeling and candidate drug screening.

The cell sorting strategy based on the use of functional markers allows the purification of hPSC-derived PAX3+/PAX7+ skeletal muscle precursors. To the inventors' knowledge, this is the first method describing the derivation and isolation of early muscle precursors with a defined phenotype.

1. An in vitro method for producing skeletal muscle precursor cells from pluripotent stem cells, comprising contacting the stem cells with a WNT agonist for a time and under conditions sufficient to induce differentiation of the stem cells into skeletal muscle precursor cells.

2. The method according claim 1, comprising the steps of
i) contacting the stem cells with the WNT agonist or GSK3β inhibitor;
ii) contacting the stem cells with a fibroblast growth factor (FGF); and
iii) allowing the cells to differentiate into stem cell progeny.
wherein step (i) precedes step (ii) by up to 4 days.

3. The method according to claim 2, wherein the WNT agonist or GSK3β inhibitor is removed prior to contacting with FGF.

4. The method according to claim 3, wherein the FGF is withdrawn after about 14 days of contact with the stem cells.

5. The method according to claim 2, wherein the FGF is FGF2.

6. The method according to claim 2 further comprising iv) culturing the stem cells in the absence of any factor for a time and under conditions to permit expression of muscle specific markers in the differentiated stem cell progeny.

7. The method according to claim 6, wherein the cell are cultured for at least 25 days, wherein day 0 corresponds to the addition of the WNT agonist or GSK3β inhibitor.

8. The method according to claim 6, wherein the muscle cell markers are individually or collectively selected from the group consisting of SIX1, SIX4, PAX3, PAX7, LBX1, MYF5 and MYOD.

9. The method according to claim 6, wherein the differentiated stem cell progeny are PAX-3⁺/PAX-⁷+ skeletal muscle precursor cells.

10. The method according to claim 2, wherein the GSK3β inhibitor is CHIR 99021.

11. The method according to claim 1, wherein the pluripotent stem cells are selected from the group consisting of inducible pluripotent stem (iPS) cells, embryonic stem (ES) cells, STAP cells, primate pluripotent stem (pPS) cells, an embryonic stem cell line or combinations of any one of these.

12. The method according to claim 2 further comprising
(i) contacting the differentiated stem cell progeny with a ligand that binds to HNK and a ligand that binds to muscle specific nicotinic acetyl choline receptor (ACHR); and
(ii) separating HNK−/ACHR+ cells from the remaining cells.

13. The method according to claim 2 further comprising
(i) contacting the differentiated stem cell progeny with a ligand that binds to HNK and a ligand that binds to muscle specific nicotinic acetyl choline receptor (ACHR);
(ii) contacting the HNK⁻/ACHR⁻ cells with a ligand that binds to C-MET and a ligand that binds to CXCR4; and
(iii) separating C-MET⁺/CXCR4⁻, C-MET⁺/CXCR4⁺ and C-MET⁻/CXCR4⁺ cells from the remaining cells.

14. The method according to claim 13, wherein the NHK⁻/ACHR⁻ cells are selected on the basis of CXCR4 expression prior to selection based on expression of C-MET.

15. The method according to claim 13, wherein step iii) comprises separating C-MET⁺/CXCR4⁻ and C-MET⁺/CXCR4⁺ cells from the remaining cells.

16. The method according to claim 13 wherein the C-MET⁺/CXCR4⁻ and C-MET⁺/CXCR4⁺ cells are PAX3⁺ and PAX7⁺.

17. Purified skeletal muscle precursor cells produced by a method according to claim 13.

18. Purified myoblasts and myocytes produced by a method according to claim 12.

19. A composition comprising purified cells according to claim 17, together with a pharmaceutically acceptable carrier or excipient.

20. Use of purified cells according to claim 17 for in vitro screening of agent(s) which are capable of modifying the function of the cells.

21. A method of treating a skeletal muscle injury, disease or disorder in a subject in need thereof, comprising administering to the subject skeletal muscle precursor cells according to claim 17.

22. The method according to claim 21, wherein the disease or disorder is selected from the group consisting of muscular dystrophy, hereditary myopathies such as congenital myopathy, distal myopathy and mitochondrial diseases, non-hereditary myopathies such as multiple myositis, dermatomyositis and myasthenia gravis, neurogenic muscular diseases, spinal amyotrophy, bulbar amyotrophy and amyotrophic lateral sclerosis.

23. A composition comprising purified cells according to claim 18, together with a pharmaceutically acceptable carrier or excipient.

24. Use of purified cells according to claim 18 for in vitro screening of agent(s) which are capable of modifying the function of the cells.
